SEQUENCE LISTING (16 sequences)

SEQ ID NO: 1 - DNA, 1878 bp, Artificial Sequence
  Synthetic sequence
atggagatcaacaaccagaaccagtgcgtgccgtacaactgcctgtccaacccgaaggagatcatcctgggcgaggagaggctggagaccggcaacaccgtggccgacatctccctgggcctgatcaacttcctgtactccaacttcgtgccgggcggcggcttcatcgtgggcctgctggagctgatctggggcttcatcggcccgtcccagtgggacatcttcctggcccagatcgagcagctgatctcccagaggatcgaggagttcgccaggaaccaggccatctccaggctggagggcctgtccaacctgtacaaggtgtacgtgagggccttctccgactgggagaaggacccgaccaacccggccctgagggaggagatgaggatacagttcaacgacatgaactccgccctgatcaccgccatcccgctgttcagggtgcagaactacgaggtggccctgctgtccgtgtacgtgcaggccgccaacctgcacctgtccatcctgagggacgtgtccgtgttcggcgagaggtggggctacgacaccgccaccatcaacaacaggtactccgacctgacctccctgatccacgtgtacaccaaccactgcgtggacacctacaaccagggcctgaggaggctggagggcaggttcctgtccgactggatcgtgtacaacaggttcaggaggcagctgaccatctccgtgctggacatcgtggccttcttcccgaactacgacatcaggacctacccgatccagaccgccacccagctgaccagggaggtgtacctggacctgccgttcatcaacgagaacctgtccccggccgcctcctacccgaccttctccgccgccgagtccgccatcatcaggtccccgcacctggtggacttcctgaactccttcaccatctacaccgactccctggccaggtacgcctactggggcggccacctggtgaactccttcaggaccggcaccaccaccaacctgatcaggtccccgctgtacggcagggagggcaacaccgagaggccggtgaccatcaccgcctccccgtccgtgccgatcttcaggaccctgtcctacatcaccggcctggacaactccaacccggtggccggcatcgagggcgtggagttccagaacaccatctccaggtccatctacaggaagtccggcccgatcgactccttctccgagctgccgccgcaggacgcctccgtgtccccggccatcggctactcccacaggctgtgccacgccaccttcctggagaggatctccggcccgaggatcgccggcaccgtgttctcctggacccacaggtccgcctccccgaccaacgaggtgtccccgtccaggatcacccagatcccgtgggtgaaggcccacaccctggcctccggcgcctccgtgatcaagggcccgggcttcaccggcggcgacatcctgaccaggaactccatgggcgagctgggcaccctgagggtgaccttcaccggcaggctgccgcagtcctactacatcaggttcaggtacgcctccgtggccaacaggtccggcaccttcaggtactcccagccgccgtcctacggcatctccttcccgaagaccatggacgccggcgagccgctgacctccaggtccttcgcccacaccaccctgttcaccccgatcaccttctccagggcccaggaggagttcgacctgtacatccagtccggcgtgtacatcgacaggatcgagttcatcccggtgaccgccaccttcgaggccgagtacgacctggagagggcccagaaggtggtgaacgccctgttcacctccaccaaccagctgggcctgaagaccgacgtgacctaa

SEQ ID NO: 2 - DNA, 5647 bp, Artificial Sequence
  Synthetic sequence
tattgcactcccttttaactgttttttattacaaaaatgccctggaaaatgcactccctttttgtgtttgtttttttgtgaaacgatgttgtcaggtaatttatttgtcagtctactatggtggcccattatattaatagcaactgtcggtccaatagacgacgtcgattttctgcatttgtttaaccacgtggattttatgacattttatattagttaatttgtaaaacctacccaattaaagacctcatatgttctaaagactaatacttaatgataacaattttcttttagtgaagaaagggataattagtaaatatggaacaagggcagaagatttattaaagccgcgtaagagacaacaagtaggtacgtggagtgtcttaggtgacttacccacataacataaagtgacattaacaaacatagctaatgctcctatttgaatagtgcatatcagcataccttattacatatagataggagcaaactctagctagattgttgagcagatctcggtgacgggcaggaccggacggggcggtaccggcaggctgaagtccagctgccagaaacccacgtcatgccagttcccgtgcttgaagccggccgcccgcagcatgccgcggggggcatatccgagcgcctcgtgcatgcgcacgctcgggtcgttgggcagcccgatgacagcgaccacgctcttgaagccctgtgcctccagggacttcagcaggtgggtgtagagcgtggagcccagtcccgtccgctggtggcggggggagacgtacacggtcgactcggccgtccagtcgtaggcgttgcgtgccttccaggggcccgcgtaggcgatgccggcgacctcgccgtccacctcggcgacgagccagggatagcgctcccgcagacggacgaggtcgtccgtccactcctgcggttcctgcggctcggtacggaagttgaccgtgcttgtctcgatgtagtggttgacgatggtgcagaccgccggcatgtccgcctcggtggcacggcggatgtcggccgggcgtcgttctgggctcatggtagatcccccgttcgtaaatggtgaaaattttcagaaaattgcttttgctttaaaagaaatgatttaaattgctgcaatagaagtagaatgcttgattgcttgagattcgtttgttttgtatatgttgtgttgagaattaattctcgaggtcctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttcaacgatggcctttcctttatcgcaatgatggcatttgtaggagccaccttccttttccactatcttcacaataaagtgacagatagctgggcaatggaatccgaggaggtttccggatattaccctttgttgaaaagtctcaattgccctttggtcttctgagactgtatctttgatatttttggagtagacaagtgtgtcgtgctccaccatgttatcacatcaatccacttgctttgaagacgtggttggaacgtcttctttttccacgatgctcctcgtgggtgggggtccatctttgggaccactgtcggcagaggcatcttcaacgatggcctttcctttatcgcaatgatggcatttgtaggagccaccttccttttccactatcttcacaataaagtgacagatagctgggcaatggaatccgaggaggtttccggatattaccctttgttgaaaagtctcaattgccctttggtcttctgagactgtatctttgatatttttggagtagacaagtgtgtcgtgctccaccatgttgacctgcaggcatgcaagcttgttaacctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgatcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggggatccgagctcatggagatcaacaaccagaaccagtgcgtgccgtacaactgcctgtccaacccgaaggagatcatcctgggcgaggagaggctggagaccggcaacaccgtggccgacatctccctgggcctgatcaacttcctgtactccaacttcgtgccgggcggcggcttcatcgtgggcctgctggagctgatctggggcttcatcggcccgtcccagtgggacatcttcctggcccagatcgagcagctgatctcccagaggatcgaggagttcgccaggaaccaggccatctccaggctggagggcctgtccaacctgtacaaggtgtacgtgagggccttctccgactgggagaaggacccgaccaacccggccctgagggaggagatgaggatacagttcaacgacatgaactccgccctgatcaccgccatcccgctgttcagggtgcagaactacgaggtggccctgctgtccgtgtacgtgcaggccgccaacctgcacctgtccatcctgagggacgtgtccgtgttcggcgagaggtggggctacgacaccgccaccatcaacaacaggtactccgacctgacctccctgatccacgtgtacaccaaccactgcgtggacacctacaaccagggcctgaggaggctggagggcaggttcctgtccgactggatcgtgtacaacaggttcaggaggcagctgaccatctccgtgctggacatcgtggccttcttcccgaactacgacatcaggacctacccgatccagaccgccacccagctgaccagggaggtgtacctggacctgccgttcatcaacgagaacctgtccccggccgcctcctacccgaccttctccgccgccgagtccgccatcatcaggtccccgcacctggtggacttcctgaactccttcaccatctacaccgactccctggccaggtacgcctactggggcggccacctggtgaactccttcaggaccggcaccaccaccaacctgatcaggtccccgctgtacggcagggagggcaacaccgagaggccggtgaccatcaccgcctccccgtccgtgccgatcttcaggaccctgtcctacatcaccggcctggacaactccaacccggtggccggcatcgagggcgtggagttccagaacaccatctccaggtccatctacaggaagtccggcccgatcgactccttctccgagctgccgccgcaggacgcctccgtgtccccggccatcggctactcccacaggctgtgccacgccaccttcctggagaggatctccggcccgaggatcgccggcaccgtgttctcctggacccacaggtccgcctccccgaccaacgaggtgtccccgtccaggatcacccagatcccgtgggtgaaggcccacaccctggcctccggcgcctccgtgatcaagggcccgggcttcaccggcggcgacatcctgaccaggaactccatgggcgagctgggcaccctgagggtgaccttcaccggcaggctgccgcagtcctactacatcaggttcaggtacgcctccgtggccaacaggtccggcaccttcaggtactcccagccgccgtcctacggcatctccttcccgaagaccatggacgccggcgagccgctgacctccaggtccttcgcccacaccaccctgttcaccccgatcaccttctccagggcccaggaggagttcgacctgtacatccagtccggcgtgtacatcgacaggatcgagttcatcccggtgaccgccaccttcgaggccgagtacgacctggagagggcccagaaggtggtgaacgccctgttcacctccaccaaccagctgggcctgaagaccgacgtgacctaagtcgacggatccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatc

SEQ ID NO: 3 - protein, 625 aa, Artificial Sequence
  Synthetic sequence
MEINNQNQCVPYNCLSNPKEIILGEERLETGNTVADISLGLINFLYSNFVPGGGFIVGLLELIWGFIGPSQWDIFLAQIEQLISQRIEEFARNQAISRLEGLSNLYKVYVRAFSDWEKDPTNPALREEMRIQFNDMNSALITAIPLFRVQNYEVALLSVYVQAANLHLSILRDVSVFGERWGYDTATINNRYSDLTSLIHVYTNHCVDTYNQGLRRLEGRFLSDWIVYNRFRRQLTISVLDIVAFFPNYDIRTYPIQTATQLTREVYLDLPFINENLSPAASYPTFSAAESAIIRSPHLVDFLNSFTIYTDSLARYAYWGGHLVNSFRTGTTTNLIRSPLYGREGNTERPVTITASPSVPIFRTLSYITGLDNSNPVAGIEGVEFQNTISRSIYRKSGPIDSFSELPPQDASVSPAIGYSHRLCHATFLERISGPRIAGTVFSWTHRSASPTNEVSPSRITQIPWVKAHTLASGASVIKGPGFTGGDILTRNSMGELGTLRVTFTGRLPQSYYIRFRYASVANRSGTFRYSQPPSYGISFPKTMDAGEPLTSRSFAHTTLFTPITFSRAQEEFDLYIQSGVYIDRIEFIPVTATFEAEYDLERAQKVVNALFTSTNQLGLKTDVT

SEQ ID NO: 4 - DNA, 24 bp, Artificial Sequence
  Synthetic sequence
aaaaatgccgcgacttgcggacgt

SEQ ID NO: 5 - DNA, 34 bp, Artificial Sequence
  Synthetic sequence
cccgccttcagtttaaactttggcatttgtggaa

SEQ ID NO: 6 - DNA, 116 bp, Artificial Sequence
  Synthetic sequence
gacacaagacatgtcatagacggagtggtaggggagggtacgcgcgagggtgaggtcgacggttcgaatactaacaaccgcgtagcgcgcgaattttgcgcgaaaaatgccgcgac

SEQ ID NO: 7 - DNA, 118 bp, Artificial Sequence
  Synthetic sequence
ttggcatttgtggaatagatcccgagtctcctacaacacaaggcaagccccggtgcatttaccctttccttgtgtttttaaactccttatcacttgaattgatgcattaggtgatagg

SEQ ID NO: 8 - DNA, 6424 bp, Artificial Sequence
  Synthetic sequence
  misc_feature (1)..(116): sequncia flanqueadora 5
  misc_feature (117)..(6306): sequncia completa do inserto
  misc_feature (6307)..(6424): sequncia flanqueadora 3
gacacaagacatgtcatagacggagtggtaggggagggtacgcgcgagggtgaggtcgacggttcgaatactaacaaccgcgtagcgcgcgaattttgcgcgaaaaatgccgcgacttgcggacgtttttaatgtactgaattaacgccgaattgctctagcattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctaattcgcttcaagacgtgctcaaatcactatttccacacccctatatttctattgcactcccttttaactgttttttattacaaaaatgccctggaaaatgcactccctttttgtgtttgtttttttgtgaaacgatgttgtcaggtaatttatttgtcagtctactatggtggcccattatattaatagcaactgtcggtccaatagacgacgtcgattttctgcatttgtttaaccacgtggattttatgacattttatattagttaatttgtaaaacctacccaattaaagacctcatatgttctaaagactaatacttaatgataacaattttcttttagtgaagaaagggataattagtaaatatggaacaagggcagaagatttattaaagccgcgtaagagacaacaagtaggtacgtggagtgtcttaggtgacttacccacataacataaagtgacattaacaaacatagctaatgctcctatttgaatagtgcatatcagcataccttattacatatagataggagcaaactctagctagattgttgagcagatctcggtgacgggcaggaccggacggggcggtaccggcaggctgaagtccagctgccagaaacccacgtcatgccagttcccgtgcttgaagccggccgcccgcagcatgccgcggggggcatatccgagcgcctcgtgcatgcgcacgctcgggtcgttgggcagcccgatgacagcgaccacgctcttgaagccctgtgcctccagggacttcagcaggtgggtgtagagcgtggagcccagtcccgtccgctggtggcggggggagacgtacacggtcgactcggccgtccagtcgtaggcgttgcgtgccttccaggggcccgcgtaggcgatgccggcgacctcgccgtccacctcggcgacgagccagggatagcgctcccgcagacggacgaggtcgtccgtccactcctgcggttcctgcggctcggtacggaagttgaccgtgcttgtctcgatgtagtggttgacgatggtgcagaccgccggcatgtccgcctcggtggcacggcggatgtcggccgggcgtcgttctgggctcatggtagatcccccgttcgtaaatggtgaaaattttcagaaaattgcttttgctttaaaagaaatgatttaaattgctgcaatagaagtagaatgcttgattgcttgagattcgtttgttttgtatatgttgtgttgagaattaattctcgaggtcctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttcaacgatggcctttcctttatcgcaatgatggcatttgtaggagccaccttccttttccactatcttcacaataaagtgacagatagctgggcaatggaatccgaggaggtttccggatattaccctttgttgaaaagtctcaattgccctttggtcttctgagactgtatctttgatatttttggagtagacaagtgtgtcgtgctccaccatgttatcacatcaatccacttgctttgaagacgtggttggaacgtcttctttttccacgatgctcctcgtgggtgggggtccatctttgggaccactgtcggcagaggcatcttcaacgatggcctttcctttatcgcaatgatggcatttgtaggagccaccttccttttccactatcttcacaataaagtgacagatagctgggcaatggaatccgaggaggtttccggatattaccctttgttgaaaagtctcaattgccctttggtcttctgagactgtatctttgatatttttggagtagacaagtgtgtcgtgctccaccatgttgacctgcaggcatgcaagcttgttaacctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgatcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggggatccgagctcatggagatcaacaaccagaaccagtgcgtgccgtacaactgcctgtccaacccgaaggagatcatcctgggcgaggagaggctggagaccggcaacaccgtggccgacatctccctgggcctgatcaacttcctgtactccaacttcgtgccgggcggcggcttcatcgtgggcctgctggagctgatctggggcttcatcggcccgtcccagtgggacatcttcctggcccagatcgagcagctgatctcccagaggatcgaggagttcgccaggaaccaggccatctccaggctggagggcctgtccaacctgtacaaggtgtacgtgagggccttctccgactgggagaaggacccgaccaacccggccctgagggaggagatgaggatacagttcaacgacatgaactccgccctgatcaccgccatcccgctgttcagggtgcagaactacgaggtggccctgctgtccgtgtacgtgcaggccgccaacctgcacctgtccatcctgagggacgtgtccgtgttcggcgagaggtggggctacgacaccgccaccatcaacaacaggtactccgacctgacctccctgatccacgtgtacaccaaccactgcgtggacacctacaaccagggcctgaggaggctggagggcaggttcctgtccgactggatcgtgtacaacaggttcaggaggcagctgaccatctccgtgctggacatcgtggccttcttcccgaactacgacatcaggacctacccgatccagaccgccacccagctgaccagggaggtgtacctggacctgccgttcatcaacgagaacctgtccccggccgcctcctacccgaccttctccgccgccgagtccgccatcatcaggtccccgcacctggtggacttcctgaactccttcaccatctacaccgactccctggccaggtacgcctactggggcggccacctggtgaactccttcaggaccggcaccaccaccaacctgatcaggtccccgctgtacggcagggagggcaacaccgagaggccggtgaccatcaccgcctccccgtccgtgccgatcttcaggaccctgtcctacatcaccggcctggacaactccaacccggtggccggcatcgagggcgtggagttccagaacaccatctccaggtccatctacaggaagtccggcccgatcgactccttctccgagctgccgccgcaggacgcctccgtgtccccggccatcggctactcccacaggctgtgccacgccaccttcctggagaggatctccggcccgaggatcgccggcaccgtgttctcctggacccacaggtccgcctccccgaccaacgaggtgtccccgtccaggatcacccagatcccgtgggtgaaggcccacaccctggcctccggcgcctccgtgatcaagggcccgggcttcaccggcggcgacatcctgaccaggaactccatgggcgagctgggcaccctgagggtgaccttcaccggcaggctgccgcagtcctactacatcaggttcaggtacgcctccgtggccaacaggtccggcaccttcaggtactcccagccgccgtcctacggcatctccttcccgaagaccatggacgccggcgagccgctgacctccaggtccttcgcccacaccaccctgttcaccccgatcaccttctccagggcccaggaggagttcgacctgtacatccagtccggcgtgtacatcgacaggatcgagttcatcccggtgaccgccaccttcgaggccgagtacgacctggagagggcccagaaggtggtgaacgccctgttcacctccaccaaccagctgggcctgaagaccgacgtgacctaagtcgacggatccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcggttaacgaattcgtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattggagcttgagcttggatcagattgtcgtttcccgccttcagtttaaactttggcatttgtggaatagatcccgagtctcctacaacacaaggcaagccccggtgcatttaccctttccttgtgtttttaaactccttatcacttgaattgatgcattaggtgatagg

SEQ ID NO: 9 - DNA, 20 bp, Artificial Sequence
  Synthetic sequence
aggtcgacggttcgaatact

SEQ ID NO: 10 - DNA, 21 bp, Artificial Sequence
  Synthetic sequence
gctagagcaattcggcgttaa

SEQ ID NO: 11 - DNA, 110 bp, Artificial Sequence
  Synthetic sequence
aggtcgacggttcgaatactaacaaccgcgtagcgcgcgaattttgcgcgaaaaatgccgcgacttgcggacgtttttaatgtactgaattaacgccgaattgctctagc

SEQ ID NO: 12 - DNA, 20 bp, Artificial Sequence
  Synthetic sequence
acaaccgcgtagcgcgcgaa

SEQ ID NO: 13 - DNA, 20 bp, Artificial Sequence
  Synthetic sequence
cggtttgcgtattggagctt

SEQ ID NO: 14 - DNA, 20 bp, Artificial Sequence
  Synthetic sequence
ggcttgccttgtgttgtagg

SEQ ID NO: 15 - DNA, 111 bp, Artificial Sequence
  Synthetic sequence
cggtttgcgtattggagcttgagcttggatcagattgtcgtttcccgccttcagtttaaactttggcatttgtggaatagatcccgagtctcctacaacacaaggcaagcc

SEQ ID NO: 16 - DNA, 25 bp, Artificial Sequence
  Synthetic sequence
tggatcagattgtcgtttcccgcct

(19) United States
(12) Patent Application Publication
(10) Pub. No.: US 2023/0135492 A1
(43) Pub. Date: May 4, 2023

(54) NUCLEIC ACID MOLECULE OF TRANSGENIC MAIZE EVENT ME240913 THAT EXPRESSES CRY1DA PROTEIN, CELL, PLANT AND TRANSGENIC SEED, USES THEREOF, PLANT PRODUCT, METHOD, KIT AND AMPLICON FOR DETECTING THE EVENT, AND METHODS TO PRODUCE A TRANSGENIC PLANT AND TO CONTROL LEPIDOPTERAN INSECT PESTS

(71) Applicants: Empresa Brasileira de Pesquisa Agropecuária - Embrapa, Brasilia (BR); Helix Sementes e Mudas Ltda., Rio Claro (BR)

(72) Inventors: Andréa ALMEIDA CARNEIRO, Brasilia (BR); Beatriz DE ALMEIDA BARROS, Brasilia (BR); Claudia TEIXEIRA GUIMARÃES, Brasilia (BR); Fernando HERCOS VALICENTE, Brasilia (BR); Marcos DE OLIVEIRA PINTO, Brasilia (BR); Meire DE CÁSSIA ALVES, Brasilia (BR); Myriam MAIA NOBRE, Brasilia (BR); Newton PORTILHO CARNEIRO, Brasilia (BR); Roberto WILLIANS NODA, Brasilia (BR); Simone MARTINS MENDES, Brasilia (BR); Sidney NETTO PARENTONI, Brasilia (BR)

(73) Assignees: Empresa Brasileira de Pesquisa Agropecuária - Embrapa, Brasilia (BR); Helix Sementes e Mudas Ltda., Rio Claro (BR)

(21) Appl. No.: 17/775,168

(22) PCT Filed: Oct. 13, 2020
(86) PCT No.: PCT/BR2020/050407

§ 371 Date: Jan. 12, 2023

(30) Foreign Application Priority Data
Nov. 6, 2019 (BR) 102019023319-2

(51) Int. Cl. A01H 1/00; A01H 6/46; A23L 7/00; C07K 14/325; C12N 15/82; C12Q 1/6813; C12Q 1/6895

(52) U.S. Cl. CPC A01H 1/00; A01H 6/46; A23L 7/00; C07K 14/325; C12N 15/82; C12Q 1/6813; C12Q 1/6895

(57) ABSTRACT

The present invention relates to a novel transgenic maize event expressing the truncated or modified Cry1Da insecticidal protein designated as Event ME240913. The invention relates to nucleic acids that are unique to Event ME240913. Primers, amplicon, methods and kits for detecting the presence of Event ME240913 are also defined. The invention further relates to maize plants containing said event, uses thereof, methods and compositions for controlling lepidopteran insect pests. The invention describes a maize event that has shown a high level of plant protection against feeding damage caused by Lepidoptera, including S. frugirperda, as well as Cry1F resistant insects. The event of the invention was shown to be highly toxic to S. frugirperda.

### FIELD OF THE INVENTION

The present invention relates to the field of plant molecular biology, plant transformation, and plant reproduction and pest control. More specifically, the invention relates to transgenic maize (Zea mays) plants resistant to lepidopteran insect pests comprising a new transgenic genotype, uses thereof, methods of controlling lepidopteran insect pests and of detecting the presence of nucleic acids that are exclusive of the transgenic maize plants in a plant product and compositions thereof.

### BACKGROUND OF THE INVENTION

Consistent advances in genetic engineering techniques have enabled the development of transgenic plants of commercial importance, containing the heterologous genes of interest, which can confer desirable traits to such plants. From among the genes of interest are genes that confer on plants resistance to herbicides, environmental stresses, diseases and invertebrate pests.

In the context of genes that code for proteins useful for controlling invertebrate pests, cry gene, derived from the Gram-positive bacterium Bacillus thuringiensis (Bt), can be mentioned. Said bacteria, which occurring naturally in several habitats, including the soil, phylloplane, grain residues, dust, water, plant matter and insects, has the innate characteristic of forming protein crystals during the stationary and/or sporulation phase. Protein crystals or delta-endotoxins, representing 20 to 30% of the total cell protein (Boucias & Pendland, 1998), can have specific insecticidal properties and be of various shapes, such as: bipyramidal, spherical, rectangular, cuboid and irregular. Bipyramidal crystals have a higher frequency of toxicity than crystals of other shapes, particularly against lepidopterans.

The mechanism of action of Cry proteins with insecticidal effect generally involves the solubilization of crystals in the midgut of the target insects, the digestive action of proteases present in the insect intestines on pro-toxins, adherence of active toxins to midgut receptors and insertion of said active Cry toxins into the apical cell membrane, creating ion channels or pores (cytolysis). These channels, when formed, can alter the insect's feeding habits and disrupt gut integrity, leading to a reduced growth and even death.

An advantage of using Cry proteins in agriculture is the selective toxicity of each of these proteins against various insect pest species, and their non-toxic nature to other vertebrate organisms such as fish, amphibians, reptiles, birds and mammals. Another advantage of Cry proteins is its relative specificity towards pest insects from different crops. Several cry genes are known. Cry1, cry2 and cry9 genes are generally active against lepidopterans; cry2, cry4A, cry10, cry11, cry17, cry19, cry24, cry25, cry27, cry29, cry30, cry32, cry39 and cry40 genes are generally active against dipterans; cry3, cry7 and cry8 genes are generally active against coleopterans; and cry5, cry12, cry13 and cry14 genes are generally active against nematodes. Commercial products containing Bt Cry proteins having insecticidal activity are currently being produced and used as biopesticides. These products represent a high percent of sales and have been used for more than 60 years mainly to control Lepidoptera and Diptera pests. Another use of such Cry proteins is their expression in transgenic plants.

Despite the use of specific Bt proteins in transgenic maize plants to control insect pests, their use has produced Lepidoptera and Coleoptera populations resistant to some of these Bt proteins (Tabashnik, B. E.; Brévault, T.; Carrière, Y. Insect resistance to genetically engineered crops: successes and failures. ISB News Report: Agricultural and Environmental Biotechnology, Jan. 2014.). One particular case is the emergence of Spodoptera frugiperda populations that are specifically resistant to Cry1F and Cry1A genes, which is most commonly found under tropical conditions. Spodoptera is the main pest found in maize-growing regions worldwide and the development of resistance to Cry1F and Cry1A proteins represents a significant challenge for maize production in these regions. Spodoptera populations in the field that were found to be resistant to Cry1F proteins were discovered (1) in 2010 in Puerto Rico (Storer, N. P.; Babcock, J. M.; Schlenz, M.; Meade, T.; Thompson, G. D.; Bing, J. W.; Huckaba, R. M. Discovery and characterization of field resistance to Bt maize: S. frugiperda (Lepidoptera: Noctuidae) in Puerto Rico. Journal of Economic Entomology, v.103, p.1031-1038, 2010.), (2) in the United States (Huang, F.; Qureshi, J. A.; Meagher JR, R. L.; Reisig, D. D.; Head, G. P.; Andow, D. A.; NI, X.; Kerns, D.; Buntin, G. D.; Niu, Y.; Yang, F.; Dangal, V. Cry1F resistance in fall armyworm S. frugiperda: single gene versus pyramided Bt maize.) and (3) in Brazil (Farias, J. R.; Andow, D. A.; Horiksoshi, R. J.; Sorgatto, R. J.; Fresia, P.; Santos, A. C.; Omoto, C. Field-evolved resistance to Cry1 F maize by S. frugiperda (Lepidoptera: Noctuidae) in Brazil. Crop Protection, v.64, p.150-158, 2014). In Brazil, S. frugiperda populations resistant to CrylAb were also found (Omoto, C.; Berbardi, O. Salmeron, E.; Sorgatto, R. J.; Dourado, P. M.; Crivellari, A.; Carvalho, R. A.; Willse, A.; Martinelli, S.; Head, G. P. Field-evolved resistance to Cry1Ab maize by S. frugiperda in Brazil. Pest Management Science, Chichester, 2016). Some of these Cry1F-resistant S. frugiperda populations develop rapidly as a result of the selective pressure from products based on transgenic plants containing a single cry1 gene active against S. frugiperda.

Due to this huge problem of the development of resistance to single Cry1 F-expressing genes in maize plants, commercial cultivars are made available, which contain a combination of two or more insecticidal genes such as cry1Ab, cry1F, vip3A, cry1A.105, cry2Ab, cry3Bb, cry34Ab, cry35Ab, mcry3A, ecry3.1Ab, and dvsnf7.

It is therefore critical to use proteins exhibiting different modes of action in the so-called pyramiding of proteins that slow down the rate of resistance development. Accordingly, there has been great interest in continuing to discover new proteins active against S. frugiperda that find no cross-resistance to those that are already resistant to Cry1 F, for example.

Gene dvsnf7 is another insect pest controlling technology that uses double-stranded RNA to inhibit genes important for insect survival. However, it has little effect on the lepidopteran order, which is extremely voracious and rapidly causes defoliation.

There is, therefore, a great need for the development of new alternative proteins, which are toxic to both wild-type insect populations and Cry1F/Cry1A resistant populations. These new proteins should be of high value for use in transgenesis strategies, being applicable in transgenesis technology and being able to control lepidopteran insect pests in transgenic crops.

One of these proteins is Cry1 Da that is produced by some Bt strains and are known to be toxic to S. frugiperda (Costa, ML, Lana, UG, Barros, EC, Paiva, L V and Valicente, FH, J of Agricultural Science, 2014, vol 6, pp128-136). However, Cry1 Da protein is described in previous reports as having a limited spectrum of toxicity against lepidopteran pests (von Frankenhuyzen, 2009) and variable toxicity for different populations of a lepidopteran species, such as S. frugiperda. For example, Monnerat et al (2006) have found that Cry1 Da protein was toxic to S. frugiperda collected in Colombia and Mexico, but was non-toxic to a population collected in Brazil (Applied and Environmental Microbiology, 2006, vol 72, p. 7029-7035).

The relatively small spectrum of activity against lepidopteran pests and its variability in terms of toxicity has been an important limitation of the use of Cry1Da. Specifically, von Frankenhuyzen (Frankenhuyzen, K. 2009. Minireview: Insecticidal activity of Bacillus thuringiensis crystal proteins. Journal of Invertebrate Pathology. 101: 1-16) has reported that from among Cry1 proteins Cry1Da is the one having the lowest spectrum of activity against a variety of lepidopteran species (toxic against only 44% of the tested species) as compared to the main Cry1A and 2Aa proteins tested (active against >80% of the tested species). Due to native Cry1Da protein limitations in terms of spectrum of activity and its variable toxicity level, researchers have been working to improve the structure of native Cry1Da protein so as it becomes more efficient against a greater spectrum of insect pests of the Lepidoptera order, more specifically corn earworm (Helicoverpa zea).

WO2007107302 describes the use of Cry1C, Cry1D or Cry1Da sequences to produce a novel chimeric protein that is potentially active against corn earworm. Likewise, documents WO2015143311 and WO2016061377 describe structural modifications in Cry1Da protein in an attempt to broaden its spectrum against other lepidopteran pests, in particular, corn earworm (Helicoverpa zea), including modifications in the native Cry1Da amino acid sequence or in fusion with other insecticidal proteins to increase insecticidal activity against a broader spectrum of lepidopteran pests. Because these experiments and findings have been focused on broadening the spectrum of Cry1Da activity against other lepidopterans, there is essentially no data available concerning toxicity of Cry1Da or its naturally occurring variants against S. frugiperda populations, including those resistant to Cry1F. The only study involving the assessment of Cry1Da expressed in transgenic maize plants has shown that this protein has a small or moderate protective effect against foliar damage caused by S. frugiperda (population not specified in the work). Furthermore, no specific data on this protein's toxicity against this pest are shown. Given the level of damage caused in the aforementioned study, it is reasonable to conclude that the transgenic plants had only a small toxic effect on the insects.

With respect to genetically modified plants, DNA constructs encoding desired proteins are individually inserted into the plant genome by genetic transformation. Current plant transformation methods primarily use Agrobacterium tumefaciens, which generally leads to a low copy number of gene constructs within the host plant genome. The gene constructs comprise a promoter, a coding region and a terminator. The promoter is the regulatory and expression-determining element both temporally and gene-especially. Generally, for a sufficiently large expression, as is the case for the production of insecticidal protein to control lepidopteran insect pests, the constructs use constitutive promoters such as the one that controls the ubiquitin gene expression in maize.

DNA construct integration into the host genome is random, and such a random insertion into the plant genome DNA can impact a gene whose product is critical to survival of the plant, rendering the resulting plant unviable. Furthermore, random insertion can target a host genome region that can negatively affect the expression of the gene of interest, regardless of the use of constitutive promoters. In other examples, overproduction of the construct's gene product has deleterious effects on the cell, leading primarily to decreased yield. Because of these potential problems, dozens (in some cases hundreds) of different events are usually produced and screened for a single event that has the desired patterns and levels of transgene expression for commercial purposes.

An event having the desired levels or patterns of transgenic expression is useful for being transferred to other genetic backgrounds of the same species through a traditional sexual crossing. There are reports of genes that, even in high and constitutive expression levels in the parent genotype, did not necessarily present the same response in other genetic backgrounds. Thus, it is desirable that, in addition to an adequate spatial and temporal expression, transformation events should present a low variation of gene expression in crosses with other genotypes (different backgrounds).

In this case, the offspring of such crosses retain the transgenic expression characteristics of the original transforming host plant. This strategy is used to ensure reliable gene expression in multiple varieties that are well adapted to the local growing conditions. This is affected by having inserted the integrated DNA into optimal locations within the genome, thus providing the best levels of temporal and spatial expression, stability across multiple generations, and across multiple genetic origins. As such, the physical genomic location of the inserted DNA becomes a key feature of the effectiveness of the resulting product and is therefore novel.

It would also be of great interest to establish a method capable of detecting the presence of a specific transformation event, in order to determine the presence of said event for testing seed quality, field release and in plants or processed samples. Such methods could also be used to determine and monitor gene segregation in progenies of sexual crosses, screening the event through crosses and detecting it in foods derived from recombinant plants. A well-known nucleic acid detection method is, but not limited to, the in vitro DNA amplification PCR (Polymerase Chain Reaction) technique using polynucleotide primers. The other method is DNA hybridization using nucleic acid probes. Detection methods can use primers or probes based on ordinary elements between different gene constructs or based on specific regions of the construct.

For the above reasons, there is a need to identify detection methods based on novel nucleic acid sequences that are unique to the transgenic maize event, useful for identifying the transgenic maize event and for detecting nucleic acids from the transgenic maize event in a plant product, as well as kits comprising the required reagents for use in detecting these nucleic acids in a plant product.

### SUMMARY

The present invention relates to a transgenic maize event, designated as ME240913 comprising a novel transgenic genotype that contains a cry1Da nucleic acid sequence optimized for expression in maize. The cry1Da coding sequence encodes a truncated variant of the native Cry1Da protein of SEQ ID NO: 3 which surprisingly confers on plants a high level of protection against leaf damage caused by several S. frugiperda populations that occur naturally in Brazil (both wild type and resistant to Cry1F). Importantly, the leaf tissue of Event ME240913 produces an unexpected high toxicity to lepidopteran insect pests, such as Noctuidae insects, particularly S. frugiperda. In addition to the cry1Da coding sequence, the present invention further provides other nucleic acids that are unique to Event ME240913, namely, the sequence of an amplicon resulting from a PCR reaction using specific primers to confirm the 5′ junction sequence, the 3′ junction sequence, 5′ and 3′ flanking sequences, and/or complements thereof. The invention further provides amplicons comprising the unique nucleic acids of Event ME240913, transgenic maize plants comprising the unique nucleic acids of Event ME240913 and seeds of the transgenic maize plants.

The present invention also relates to methods for producing a transgenic maize plant comprising the unique nucleic acids of the invention by sexually crossing a first parental maize plant with a second parental maize plant to produce a plurality of first-generation progeny plants, wherein at least one of said parental plants comprises a nucleic acid unique to Event ME240913, selecting a first-generation progeny plant that is resistant to infestation by lepidopteran insect pests, self-pollinating the first-generation progeny plant to produce a plurality of second-generation progeny plants, and selecting, from among the second-generation progeny plants a plant that is resistant to lepidopteran insect pests.

The present invention further describes methods for controlling lepidopteran insect pests, such as those of the Noctuidae family, particularly S. frugiperda. Maize plants comprising Event ME240913 and methods of the present invention are effective for controlling specific lepidopteran insect-pest populations, such as S. frugiperda, which have become resistant to plants that express Cry1F protein.

Methods for producing hybrid maize seeds are also disclosed. Such methods comprise the steps of planting seeds of a first congenital maize line comprising at least one nucleotide sequence unique to Event ME240913 and of seeds of a second congenital line having a different genotype, growing maize plants resulting from said seeds planted until flowering time, emasculation of the flowers of plants of one of the congenital maize lines, sexual crossing of the two different congenital lines with each other, and harvesting of the hybrid seed thus produced.

A seed sample, and accordingly, seed-grown maize plants, comprising nucleic acids unique to Event ME240913 was deposited with the American Type Culture Collection (ATCC) under accession number PTA-126224. The transgenic maize plants of the invention exhibit essentially all of the corresponding morphological and physiological characteristics of the non-transgenic isogenic maize plants in addition to those conferred on the maize plants by the novel genotype of the invention. Plant products and extracts from ME240913 maize plants, tissues and seeds are also provided by the present invention.

The present invention further provides methods of introgressing Event ME240913 into maize lines by sexually crossing plants containing Event ME240913, such as, for example, but not limited to plants obtained from seeds deposited with the American Type Culture Collection (ATCC) under accession number PTA-126224.

In accordance with the present invention, Event ME240913 can be combined with other maize transgenic events by methods known in the art, such as gene pyramiding. The teaching that ME240913 produces a high level of control against plant damage caused by various Brazilian S. frugiperda populations (including Cry1F-resistant insects) establishes that the new event is toxic to Cry1F-resistant S. frugiperda populations in Brazil. These results also indicate that Event ME240913 acts through a different mechanism of action than Cry1F and, therefore, the maize event will be very effective when combined with other insecticidal genes incorporated into the plant using gene pyramiding. Event ME240913 also expresses the herbicide resistance pat (bar) gene. Therefore, Event ME240913 can also be used to reduce the herbicide resistance rate using gene pyramiding.

Examples of gene pyramiding include, but are not limited to, herbicide and insect pest resistance events. Using such methods, Event ME240913 can be combined, for example, with events containing one or more genes selected from pat gene, cp4 epsps (5-enolpyruvyl-shikimate-3-phosphate synthase) gene, cry1Ab gene, cry1F gene, vip3A gene, cry1A.105 gene, cry2Ab gene, cry3Bb gene, cry34Ab gene, cry35Ab gene, mcry3A gene, ecry3.1Ab, dvsnf7 gene, and amy797E gene.

The present invention further provides a pair of polynucleotide primers comprising a first polynucleotide primer and a second polynucleotide primer, which function together in the presence of a DNA template of Event ME240913 in a sample to produce a diagnostic amplicon for Event ME240913, wherein the first polynucleotide primer comprises a portion of the 5′ flanking sequence, the 3′ flanking sequence, and/or complements thereof, wherein the second polynucleotide primer comprises a portion of a specific insert sequence, or complements thereof, and wherein said maize Event ME240913 has a specific sequence containing the event insert and flanking sequences in maize genome.

The present invention relates to transgenic maize plants as well as cells and tissues thereof, which comprise a nucleic acid molecule of the invention.

In one embodiment, the transgenic maize plant is resistant to lepidopteran insect pests. In a most preferred embodiment, the transgenic maize plant is highly resistant to leaf damage caused by naturally occurring S. frugiperda populations found in maize producing regions in Brazil.

In another embodiment, the transgenic maize plant is highly toxic to S. frugiperda, leading to 100% mortality when fed with fresh leaf tissue. In another embodiment, the transgenic maize plant is a plant highly toxic to S. frugiperda, as evidenced by high mortality and morbidity of S. frugiperda after dilution of freeze-dried leaves of Event ME240913 at a 1:25 ratio (weight/weight) with artificial diet. High toxicity of freeze-dried plant leaf tissue of Event ME240913 shows that said event is very efficient in controlling S. frugiperda and is useful for pyramiding several insect controlling genes.

Maize seeds comprising a nucleic acid molecule of the invention are also disclosed. In one embodiment, the maize seeds are deposited with the American Type Culture Collection under accession number PTA-126224 and are used to produce transgenic maize plants.

Furthermore, the present invention relates to a maize plant product of Event ME240913, tissue or seed thereof, which comprises a nucleotide sequence of the present invention or complements thereof, and wherein the sequence is detectable in the plant product via amplification of the nucleic acid or a method of nucleic acid hybridization.

The invention further contemplates plant products, such as, but not limited to, corn grain, forage, corn flour, corn meal, corn syrup, corn oil, corn starch, and cereals made in whole or in part of corn-derived products.

The present invention further provides kits for detecting nucleic acids that are unique to Event ME240913, which comprise at least one nucleic acid molecule that is a primer or probe comprising a nucleic acid sequence comprising a specific sequence, and complements thereof, wherein said primer or probe is diagnostic for the presence of nucleic acid sequences unique to Event ME240913 in the sample by amplifying or hybridizing a target nucleic acid sequence in a sample followed by detection of the amplicon or hybridization to the target sequence.

Further disclosed are methods for detecting the presence of at least one nucleic acid molecule that is unique to Event ME240913 in a sample comprising maize nucleic acids, wherein said methods comprise the following steps: contacting the sample with a pair specific primers of the present invention, performing a nucleic acid amplification reaction so as to produce an amplicon, and detecting said amplicon, wherein the amplicon comprises nucleic acid sequences unique to Event ME240913, or complements thereof, and wherein said maize Event ME240913 has a specific sequence containing the event insert and flanking sequences in maize genome.

Another method for detecting the presence of a nucleic acid molecule that is unique to Event ME240913 in a sample comprising nucleic acids from maize comprises the steps of: contacting the sample with a probe that hybridizes under high stringency conditions to the DNA of Event ME240913 and does not hybridize under high stringency conditions with the DNA of a control maize plant, wherein the probe comprises a nucleotide sequence unique to Event ME240913, and complements thereof, subjecting both the sample and probe to high stringency hybridization conditions, and detecting hybridization of the probe to the nucleic acid molecule, wherein said maize Event ME240913 has a specific sequence containing the event insert and flanking sequences in the maize genome.

Further, there is disclosed a method for controlling lepidopteran insect pests in maize plants, wherein the maize plants comprise a nucleic acid molecule comprising nucleic acid sequences unique to Event ME240913, or complements thereof, wherein said methods comprise the steps of planting seeds obtained from a plant comprising said nucleic acid sequences unique to Event ME240913 in a growing area of maize plants susceptible to lepidopteran insect pests.

Moreover, there are described methods for controlling lepidopteran insect pests in maize plants, wherein the maize plants contain truncated and modified Cry1Da protein produced by expressing unique sequences of a nucleic acid sequence unique to Event ME240913, leading to increased expression levels of the truncated and modified Cry1Da protein in the leaves, which levels are highly protective against leaf damage caused by populations that occur naturally in Brazil and are also highly toxic against S. frugiperda, as seen in the high mortality rates found in studies carried out.

Further disclosed is the use of a plant, plant cell, plant part or seed comprising Event ME240913 for crossing with a second plant, regenerating a plant, planting or growing a field of plants, or producing a plant product.

These and other aspects of the invention will become more apparent from the detailed description below.

### DESCRIPTION OF SEQUENCES IN THE SEQUENCE LISTING

SEQ ID NO: 1 is the nucleotide sequence encoding truncated Cry1Da protein optimized for expression in maize present in Event ME240913.

SEQ ID NO: 2 is the nucleic acid sequence of the full-length transgene construct of Event ME240913 comprising, in the following order: the 3′ UTR terminator region of Tvsp gene; the phosphinothricin acetyl transferase gene coding region (bar), a translational enhancer region (tev); a duplicated CaMV 35S gene promoter region, ubiquitin (ubi) gene promoter region; codon-optimized cry1Da gene nucleic acid sequence (SEQ ID NO: 1); and a 3′ UTR terminator region of the nopaline synthase gene.

SEQ ID NO: 3 is the amino acid sequence of truncated Cry1Da protein expressed by Event ME240913.

SEQ ID NO: 4 is the 5′ junction sequence.

SEQ ID NO: 5 is the 3′ junction sequence.

SEQ ID NO: 6 is the 5′ flanking sequence.

SEQ ID NO: 7 is the 3′ flanking sequence.

SEQ ID NO: 8 is the nucleotide sequence comprising the 5′ flanking sequence (nucleotides 1-116), the full-length insert sequence (nucleotides 117-6306), and the 3′ flanking sequence (nucleotides 6307-6424) of Event ME240913.

SEQ ID NO: 9 is a forward primer sequence useful for amplifying the 5′ junction sequence.

SEQ ID NO: 10 is a reverse primer sequence useful for amplifying the 5′ junction sequence.

SEQ ID NO: 11 is an illustrative PCR amplicon sequence obtained using primers specific to confirm the 5′ junction sequence.

SEQ ID NO: 12 is a probe sequence useful for detecting the 5′ junction sequence.

SEQ ID NO: 13 is a forward primer sequence useful for amplifying the 3′ junction sequence.

SEQ ID NO: 14 is a reverse primer sequence useful for amplifying the 3′ junction sequence.

SEQ ID NO: 15 is an illustrative PCR amplicon sequence obtained using primers specific to confirm the 3′ junction sequence.

SEQ ID NO: 16 is a probe sequence useful for detecting the 3′ junction sequence.

### BRIEF DESCRIPTION OF THE FIGURES

FIG. 1 is a representative diagram of the insert of Event ME240913 and the respective nucleotide sequences that define said event, including 5′ and 3′ junction and flanking sequences of the maize genome.

FIG. 2 is a diagram depicting gene constructs Ubi::cry1Da::NOS e 2x35S::bar::Tvsp of Event ME240913 insert inserted into Hind III and EcoRI enzyme sites of binary vector pTF101.1, between the TDNA right and left borders.

FIG. 3 shows the result of the evaluation of Event ME240913 relative to S. frugiperda control using fresh maize leaves for a period of 5 days. Fresh leaf samples of non-GMO maize (a) and maize genetically modified with Cry1Da (b) are shown.

FIG. 4 shows the results of assays of S. frugiperda exposure to fresh leaf tissue from two genetic backgrounds containing Event ME240913. S. frugiperda survival (%) was assessed up to 3 days after exposure of newly hatched larvae to fresh control maize leaves and maize leaves expressing Event ME240913 under two different genetic backgrounds (hybrid and RC1F1) and is shown.

FIG. 5 shows the survival rate of newly hatched larvae after 14 days of exposure to leaves lyophilized at 1:25 dilution in artificial control diet and Event ME240913.

FIG. 6 shows the sizes of live caterpillars after 7 (A) and 10 (B) days of exposure to lyophilized leaf tissue of Event ME240913 diluted at 1:25 in the artificial diet as compared to the control diet.

FIG. 7 shows the damage level result on leaves of control maize plants (cony) and Event ME240913 (GMO) in plots infested by six different S. frugiperda populations.

FIG. 8 shows photographs of damage to control maize plants (left) and Event ME240913 maize plants (right) in a field infested by S. frugiperda populations from Palotina/PR (8A), Rondonópolis/MT (8B), Rondonópolis+Campo Verde/MT (8C), Paracatu/MG (8D), Sete Lagoas/MG (8E), and Ivatuba/PR (8F).

FIG. 9 refers to a graph showing the injury score (±CI, P=0.05) caused by S. frugiperda infestation in Oak scale, 1970. Treatment 1—transgenic maize comprising the codon-optimized cry1Da nucleic acid sequence of the present invention (SEQ ID NO: 1)+Cry1F resistant caterpillar population; Treatment 2=Non-transgenic L3 maize strain+Cry1F resistant caterpillar population; Treatment 3=transgenic maize comprising the codon-optimized cry1Da nucleic acid molecule of the present invention (SEQ ID NO: 1)+Population of susceptible caterpillars; Treatment 4=Non-transgenic L3 maize strain+Population of susceptible caterpillars.

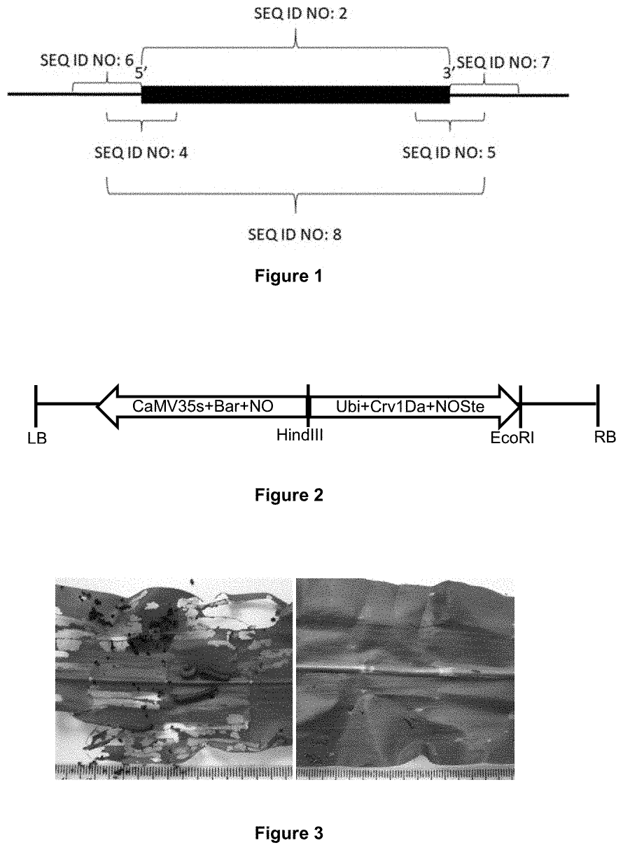

### DETAILED DESCRIPTION OF THE INVENTION

Unless otherwise defined, all terms used in the art, annotations and other scientific terminologies used herein are intended to have the meanings usually understood by those skilled in the art in the field of the present invention. In some instances, terms having the commonly understood meanings are defined in the present document for the purpose of bringing clarity and/or for prompt reference, and inclusion of such definitions in the instant document should not necessarily be interpreted as representing a substantial difference relative to what is usually understood in the state of the art.

The techniques and procedures described or referred to in the present document are generally well understood and employed using conventional methodology by those skilled in the art. As appropriate, processes involving the use of commercially available kits and reagents are generally carried out in accordance with protocols and/or parameters defined by the manufacturer, unless otherwise indicated.

It is worth mentioning that the present invention, where appropriate, is not limited to the methodology, protocols, cell line, genera or animal species, constructs and specific reagents as described, which, obviously, may vary. In addition, the terminology used in the present document is only for the purpose of describing examples of specific embodiments thereof, and is not intended to limit the scope of the present invention.

Throughout the instant document, singular forms “a” and “the” or singular forms of any term or expression, include references to the plural, unless the context clearly dictates otherwise.

Throughout the instant document, the word “comprises”, and any variations thereof such as “comprising” or “comprise” should be interpreted as “open terms”, which may imply the inclusion of additional elements or groups of elements, which were not explicitly mentioned, not having a limitative character.

Throughout the present document, the word “consists”, and any variations such as “consist” or “consisting”, should be interpreted as “closed terms”, and may not imply the inclusion of additional elements or groups of elements that were not explicitly described, having a limitative character.

Throughout the instant document, the exact values or ranges of exact values provided with respect to a particular factor, amount, concentration or particular preference should be interpreted as also providing corresponding values or ranges of approximate values, such as through the expression “about”.

Throughout the instant document, words and expressions such as “preferably”, “particularly”, “for example”, “such as”, “as”, “more particularly” and the like, and variations thereof, must be interpreted as entirely optional characteristics, preferred embodiments or possible non-exhaustive examples, without limiting the scope of the invention.

Throughout the instant document, words and expressions such as “nucleic acids”, “nucleotides” and the like should be interpreted as naturally occurring, synthetic or artificial nucleic acids or nucleotides. They comprise deoxyribonucleotides (DNA) or ribonucleotides (RNA) or any nucleotide analog and polymers or hybrids thereof in sense or antisense configuration, being either single-stranded or double-stranded. Unless otherwise stated, a specific nucleic acid sequence also implicitly encompasses conservatively modified variants thereof (for example, degenerate codon substitutions) and complementary sequences, as well as the sequences explicitly indicated. The term “nucleic acid” is used interchangeably in the present document with the terms “gene”, “cDNA”, “mRNA”, “oligonucleotide”, “nucleic acid molecule” or “primer”.

The expressions “nucleic acid molecule”, “nucleic acid sequence” and the like refer to a polymer of single-stranded or double-stranded DNA or RNA bases, read from the 5′ to the 3′ end. It includes chromosomal DNA, self-replicating plasmid, infectious DNA or RNA polymers that play a mainly structural role, among others. They also refer to a consecutive list of abbreviations, letters, characters or words representing nucleotides or genes, as usually used in the technical field of the present invention.

As used herein, the term “amplified” means the construction of multiple copies of a nucleic acid molecule or multiple copies complementary to the nucleic acid molecule using at least one of the nucleic acid molecules as a template. Amplification systems include, but are not limited to, the Polymerase Chain Reaction (PCR) system, ligase chain reaction (LCR) system, nucleic acid sequence-based amplification (NASBA, Cangene, Mississauga, Ontario), Q-Beta Replicase systems, transcription-based amplification system (TAS), and strand displacement amplification (SDA). See, for example: Diagnostic Molecular Microbiology: Principles and Applications, D. H. Persing, et al., Ed., American Society for Microbiology, Washington, D.C. (1993). The amplification product is described as an amplicon.

A “coding sequence” is a nucleic acid sequence that is transcribed into RNA such as mRNA, rRNA, tRNA, snRNA, sense RNA or antisense RNA. Preferably, the RNA is then translated in an organism to produce a protein.

Throughout the instant document, words and expressions such as “sequence similarity”, “identity” and the like, with respect to another sequence, should be interpreted as the percentage of nucleotides in the sequence that is identical to the nucleotides in another sequence, after alignment of sequences and the introduction of gaps, if necessary, to achieve the maximum percentage of sequence identity. According to the present invention, the phrase “at least 70% similarity”, for example, is defined as 70 to 100% similarity or identity. Preferably, the percent similarity is at least 75%, at least 80%, at least 85%, at least 90%, at least 95% or 100%.

A “gene” is a defined region that is located within a genome and which, despite the above-mentioned coding sequence, may comprise other sequences, primarily regulatory nucleic acid sequences that are responsible for the control of expression, i.e., transcription and translation of the coding region. A gene may also contain other 5′ and 3′ untranslated sequences and termination sequences. Other elements that may be present are, for example, introns.

“Gene of interest” refers to any gene which, when transferred to a plant, confers on the plant a desired trait, such as antibiotic resistance, virus resistance, insect resistance, disease resistance, or resistance to other pests, herbicide tolerance, improved nutritional value, improved performance in an industrial process, or altered reproductive capacity.

“Genotype” as used herein is the genetic material inherited from the parent maize plants. Genotype ME240913 refers to the heterologous genetic material transformed within the plant genome as well as the genetic material flanking the insert sequence.

A “heterologous” nucleic acid sequence is a nucleic acid sequence not naturally associated with a host cell into which it is introduced, including the non-naturally occurrence of multiple copies of a nucleic acid sequence.

A “homologous” nucleic acid sequence is a nucleic acid sequence naturally associated with a host cell into which it is introduced.

“High toxicity” of maize leaf tissue refers to the ability of leaf tissue samples to cause 100% mortality of lepidopteran species, such as S. frugiperda within 7 days of leaf tissue exposure. Another part of the definition of “high toxicity” is the ability of dry leaf tissue diluted at 1:25 with conventional maize leaf tissue to cause >95% mortality and morbidity within 14 days of exposure to the leaf tissue diet.

“Operatively linked” refers to the association of nucleic acid sequences into a single nucleic acid fragment so that the function of one affects the function of the other. For example, a promoter is operatively linked to a coding sequence or functional RNA when the latter is capable of affecting the expression of the coding sequence or functional RNA (i.e., when the coding sequence or functional RNA is under the transcriptional control of the promoter). Coding sequences in sense or antisense orientation can be operatively linked to regulatory sequences.

“Plant protection”, as used herein, refers to the ability of the intact maize plant to resist foliar damage caused by susceptible lepidopteran pests, including, but not limited to, protection against leaf damage caused by S. frugiperda. Protection can be observed by examining plants or plant photographs in order to compare the damage found in control maize plants with that observed in Cry1Da expressing maize plants. “Plant protection”, as used herein, also refers to the ability of the intact plant to resist damage from susceptible lepidopteran pests, including but not limited to the use of standard and accepted methods of classifying plant damage.

“Primers” as used herein are isolated nucleic acids that are annealed to a complementary target DNA strand by nucleic acid hybridization to form a hybrid between the primer and the target DNA strand, and then extended along the target DNA strand by a polymerase, such as DNA polymerase. Pairs or sets of primers can be used to amplify a nucleic acid molecule, for example, via Polymerase Chain Reaction (PCR) or other conventional methods of nucleic acid amplification.

A “probe” is an isolated nucleic acid to which a conventional detectable label or reporter molecule, such as a radioactive isotope, ligand, chemiluminescent agent or enzyme, is bound. Such a probe is complementary to a strand of a target nucleic acid, in the case of the present invention, to a strand of genomic DNA from maize Event ME240913. DNA from Event ME240913 can be from a maize plant or from a sample that includes DNA from Event ME240913. Probes according to the present invention include not only ribonucleic or deoxyribonucleic acids, but also polyamides and other probe materials that specifically bind to a target DNA sequence and can be used to detect the presence of that target DNA sequence.

Primers and probes are generally between 10 and 15 nucleotides or more in length. Primers and probes can also be at least 20 nucleotides or more in length, or at least 25 nucleotides or more in length, or at least 30 nucleotides or more in length. Such primers and probes specifically hybridize to a target sequence under high stringency hybridization conditions. Primers and probes according to the present invention may have a full-length sequence complementary to the target sequence, although probes differing from the target sequence and which retain the ability to hybridize to the target sequences may be designed by conventional methods.

“Stringent conditions” or “stringent hybridization conditions” include references to conditions under which a probe will hybridize to its target sequence to a greater detectable degree than to other sequences. Stringency conditions depend on the target sequence and will differ depending on the polynucleotide structure. By controlling the hybridization stringency and/or washing conditions, target sequences 100% complementary to the probe (homologous probe) can be identified. Alternatively, the stringency conditions can be adjusted to allow for some mismatch in the sequences so that lower degrees of similarity are detected (heterologous probe). Longer sequences specifically hybridize at higher temperatures. An extensive guide to nucleic acid hybridization is found in Tijssen (1993) Laboratory Techniques in Biochemistry and Molecular Biology-Hybridization with Nucleic Acid Probes, Part I, Chapter 2 “Overview of principles of hybridization and the strategy of nucleic acid probe assays”, Elsevier: New York; and Current Protocols in Molecular Biology, Chapter 2, Ausubel et al., Eds., Greene Publishing and Wiley-Interscience: New York (1995), and also Sambrook et al. (2001) Molecular Cloning: A Laboratory Manual (5^{th} Ed. Cold Spring Harbor Laboratory, Cold Spring Harbor, N.Y.).

Specificity is typically a function of post-hybridization washes, the ionic strength and temperature of the final wash solution being the critical factors. In general, high stringency hybridization and washing conditions are selected to be approximately 5° C. below the thermal melting point (Tₘ) for the specific sequence at a defined ionic strength and pH. Tₘ is the temperature (under defined ionic strength and pH) at which 50% of the target sequence hybridizes to a perfectly matched probe. Typically, under high stringency conditions a probe will hybridize to its target sequence, but not to other sequences.

An example of high stringency hybridization conditions for hybridizing complementary nucleic acids having more than 100 complementary residues in a Southern Blot or Northern Blot filter is 50% formamide plus 1 mg heparin at 42° C., hybridization being carried out overnight. An example of very high stringency wash condition is 0.15M NaCl at 72° C. for approximately 15 minutes. An example of high stringency wash condition is a 0.2×SSC wash at 65° C. for 15 minutes (see, Sambrook, infra, for a description of the SSC buffer).

A good example of hybridization conditions for the present invention include hybridization in 7% SDS, 0.25M NaPO₄ pH 7.2 at 67° C. overnight, followed by two washes in 5% SDS, 0.20M NaPO₄ pH 7.2 at 65° C. for 30 minutes each wash, and two washes in 1% SDS, 0.20 M NaPO₄ pH 7.2 at 65° C. for 30 minutes each wash. A good example of a medium stringency wash for a duplex of, for example, more than 100 nucleotides is 1× SSC at 45° C. for 15 minutes. A good example of a low stringency wash for a duplex of, for example, more than 100 nucleotides is 4-6× SSC at 40° C. for 15 minutes.

For probes of approximately 10 to 50 nucleotides, high stringency conditions typically involve salt concentrations of less than approximately 1.0 M Na ion, typically concentrations of approximately 0.01 to 1.0 M Na ion (or other salts) at pH 7.0 to 8.3, and the temperature is typically at least about 30° C. High stringency conditions can also be achieved by the addition of destabilizing agents such as formamide. Generally, a signal to noise ratio 2×(or greater) than that observed for an unrelated probe in the specific hybridization assay indicates detection of a specific hybridization. Nucleic acids that do not hybridize to each other under high stringency conditions are still substantially identical if the proteins they encode are substantially identical. This occurs, for example, when a nucleic acid copy is created using the maximum codon degeneracy allowed by the genetic code.

The following are good examples of sets of hybridization/washing conditions that can be used to hybridize nucleotide sequences that are substantially identical to the reference nucleotide sequences of the present invention: a reference nucleotide sequence preferably hybridizes to the nucleotide sequence in 7% Sodium Dodecyl Sulfate (SDS), 0.5M NaPO₄, 1 mM EDTA at 50° C. with washing in 2×SSC, 0.1% SDS at 50° C., more preferably in 7% sodium dodecyl sulfate (SDS), 0.5 M NaPO₄, 1 mM EDTA at 50° C. with washing in 1×SSC, 0.1% SDS at 50° C., most preferably in 7% sodium dodecyl sulfate (SDS), 0.5 M NaPO₄, 1 mM EDTA at 50° C. with washing in 0.5×SSC, 0.1% SDS at 50° C., preferably in 7% sodium dodecyl sulfate (SDS), 0.5 M NaPO₄, 1 mM EDTA at 50° C. with washing in 0.1×SSC, 0.1% SDS at 50° C., more preferably in 7% sodium dodecyl sulfate (SDS), 0.5M NaPO₄, 1 mM EDTA at 50° C. with washing in 0.1×SSC, 0.1% SDS at 65° C. The sequences of the present invention can be detected using all of the above conditions. For the purposes of defining the invention, high stringency conditions are used.

Throughout this document, words and expressions such as “promoter”, “promoter sequence” and the like should be interpreted as a DNA sequence that, once operatively linked to a nucleotide sequence of interest, is able to control transcription of the nucleotide sequence of interest into RNA. A promoter is located 5′ (or upstream) of the site of initiation of transcription of a nucleotide sequence of interest whose mRNA transcription it controls and provides a site for specific binding of RNA polymerase and other transcription factors for transcription to start. It may include other regulatory sequences known to a person skilled in the art. According to the present invention, the promoter can be heterologous or homologous to the respective cell or host. A nucleic acid sequence is “heterologous” to an organism or a second nucleic acid sequence if it originates from a different species or, if from the same species, it is modified from its original form.

As used herein, the term “unique” to Event ME240913 means distinctive features of Event ME240913. Thus, nucleic acids unique to Event ME240913 are not found in maize plants other than ME240913.

As used herein, the term “maize” refers to the species Zea mays and includes all plant varieties that can be reproduced with maize, including wild-type maize species.

“Detection Kit”, as used herein, refers to a kit of parts useful in the detection of the presence or absence of ME240913 plant unique nucleic acids in a sample, where the kit comprises the nucleic acid probes and/or primers of the present invention, which specifically hybridize under high stringency conditions to a target DNA sequence, and other materials required to enable nucleic acid amplification or hybridization methods.

Throughout the present document, the term “transformation” and the like should be interpreted as a process for introducing heterologous DNA into a cell, plant tissue or plant. It can take place under natural or artificial conditions, such as using several methods well known in the art, in a prokaryotic or eukaryotic host cell. The method is usually selected based on the host cell to be transformed and may include, but is not limited to, viral infection, electroporation, lipofection, particle bombardment (biobalistic) and Agrobacterium-mediated methods.

Throughout the present document, the term “transgene” should be interpreted as any nucleic acid sequence that is introduced into a cell through experimental manipulations, being integrated into the genome or not. A transgene can be an “endogenous DNA sequence”, or an “exogenous DNA sequence” (i.e., “heterologous”). The term “endogenous DNA sequence” refers to a nucleotide sequence that is naturally found in the cell into which it is introduced. The term “exogenous DNA sequence” refers to a nucleotide sequence that is not naturally found in the cell into which it is introduced. The term “transgenic” in reference to a transformed organism, means an organism transformed with a recombinant DNA molecule that preferably comprises a suitable promoter operably linked to a DNA sequence of interest.

Throughout the present document, the term “vector” should be interpreted as a construct containing a DNA sequence that is operably linked to one or more suitable control sequences capable of leading to the expression of said DNA sequence in a suitable host. Such control sequences include a promoter to perform transcription, an optional operator sequence for controlling such transcription, a sequence coding for the suitable mRNA binding sites to the ribosome, and sequences that control the end of transcription and translation, for example.

Several vectors are suitable for carrying out the present invention. These vectors can be replicated autonomously in the host organism or be replicated by the chromosome. The vector can also be a plasmid. According to the present document, the terms “plasmid” and “vector” are sometimes used interchangeably. Preferably, the vector according to the present invention comprises the cry1 Da nucleic acid molecule comprising the nucleic acid sequence of SEQ ID NO: 1, as defined herein.

As used herein the term transgenic “event” refers to a recombinant plant produced by transforming and regenerating a plant tissue or cell with heterologous DNA, for example, an expression cassette that includes a gene of interest. The term “event” refers to the original transformant and/or the transformant progeny that includes the heterologous DNA. The term “event” also refers to progeny produced by a sexual between the transformant and another maize variety. Furthermore, even after repeated back-crossing to a recurrent parent, the inserted DNA and the flanking DNA from the transformed parent are present in the progeny of the cross at the same chromosomal location. The term “event” also refers to DNA from the original transformant comprising the inserted DNA and flanking sequence immediately adjacent to the inserted DNA that would be expected to be transferred to a progeny that receives inserted DNA including the transgene of interest as the result of a sexual cross of one parental line that includes the inserted DNA (e.g., the original transformant and progeny resulting from selfing) and a parental line that does not contain the inserted DNA. Usually, plant tissue transformation generates multiple events, each representing the insertion of a DNA construct into a different location in a plant cell genome. Based on transgene expression or other desirable traits, a particular event is selected. Accordingly, the terms “Event ME240913” and “event” can be used interchangeably.

An insect resistant ME240913 maize plant can be bred by first sexually crossing a first parental maize plant consisting of a maize plant grown from the transgenic ME240913 maize plant, such as an ME240913 maize plant grown from the seed deposited with the ATCC under accession number: PTA-126224, and the progeny thereof derived from transformation with the expression cassettes of the embodiments of the present invention that confers lepidopteran insect pest resistance, with a second parental maize plant that may or may not show resistance to lepidopteran insect pests, thus producing a plurality of first first-generation progeny plants; and then selecting a first first-generation plant that is resistant to lepidopteran insect pests; and selfing the first-generation progeny plant, thereby producing a plurality of second-generation progeny plants; and then selecting from the second-generation progeny plants those plants that are resistant to lepidopteran insect pests. These steps can further include the backcrossing of the first-generation lepidopteran insect pest resistant plant or the second-generation lepidopteran insect pest resistant plant with the second parental maize plant or a third parental maize plant, thereby producing a maize plant that is resistant to lepidopteran insect pests. Such methods can be used for the introgression of Event ME240913 into maize lines as well as for pyramiding Event ME240913 with other transgenic events.

Throughout the instant document, the expressions “host cell”, “host organism” and the like should be interpreted as being the specific host organism or the specific target cell, but also as being the progeny or potential progeny of those organisms or cells. Since due to mutation or environmental effects certain modifications may appear in successive generations, these descendants need not necessarily be identical to the parental cell. However, they are still included in the scope of protection of the present invention. According to the present invention, host cells can be prokaryotic or eukaryotic. Preferably, the host cell according to the present invention is a plant host cell. Preferably, it comprises a nucleic acid sequence that is unique to Event ME240913, which is selected from SEQ ID NO: 4, SEQ ID NO: 5 and complements thereof.

Throughout the present document, words and expressions such as “transgenic plant cell”, “transgenic plant” and the like should be interpreted as cells or plants having and preferably expressing a transgene through experimental manipulations, and further refer to the progeny of a transgenic plant and subsequent plant generations, as above.

Throughout the present document, the term “plant” and the like should be interpreted as being the plant organism in whole or in part. “Part” in this context means plant cells and tissues, organs and parts of plants in all their manifestations, such as seeds, leaves, anthers, fibers, tubers, roots, root hair, stems, embryos, calluses, cotyledons, petioles, collected material, plant tissue, reproductive tissue and cell cultures. The transgenic plants according to the present invention can be generated and selfed or crossed with other individuals in order to obtain additional transgenic plants. Transgenic plants can also be obtained by vegetative propagation of transgenic plant cells.

Throughout this document, words and expressions such as “pest”, “lepidopteran insect pests” and the like shall be interpreted as insects of the order Lepidoptera, including, but not limited to, the families Papilionidae, Pieridae, Lycaenidae, Nymphalidae, Danaidae, Satyridae, Hesperiidae, Sphingidae, Saturniidae, Geometridae, Arctiidae, Noctuidae, Lymantriidae, Sesiidae, Crambidae and Tineidae, more particularly noctuids Spodoptera sp., particulary S. frugiperda (Noctuidae) and cambids Diatraea sp., particularly D. saccharalis (Crambidae).

One of the embodiments of the present invention relates to a method of controlling lepidopteran insect pests in crop plants, including but not limited to caterpillars. Any method of controlling lepidopteran insect pests in crop plants is included within the scope of the present invention, not being of particular relevance for achieving the embodiments of the invention, as long as the crop plants according to the present invention comprise at least one nucleic acid sequence that it unique to Event ME240913, which is selected from SEQ ID NO: 4, SEQ ID NO: 5, and complements thereof, wherein the method preferably comprises planting seeds obtained from a plant comprising at least one nucleic acid sequence that is unique to Event ME240913, as defined herein, in a cultivation area of crop plants susceptible to lepidopteran insect pests.

The “Cry1Da” class of proteins further comprises homologues thereof. “Homologous” means that the recited protein or polypeptide bears a defined relationship to other members of the Cry1Da class of proteins.

The present invention relates to a genetically improved maize strain that produces a truncated Cry1Da protein that is modified to control lepidopteran insect pests. The invention is particularly designed for a transgenic maize event designated as ME240913 comprising a new genotype, as well as compositions and methods for detecting nucleic acids unique to Event ME240913 in a biological sample. The invention is further designed for maize plants comprising the ME240913 genotype, transgenic seeds of the maize plants, and methods of producing a maize plant comprising ME240913 genotype by crossing a selfed maize comprising ME240913 genotype or another maize line. Maize plants comprising the ME240913 genotype of the invention are useful in controlling lepidopteran insect pests including, but not limited to, the Noctuidae and/or Crambidae family, preferably S. frugiperda and D. saccharalis. Maize plants from Event ME240913 show plant protection against sensitive lepidopteran pests, including but not limited to wild-type Cry1F-resistant and Cry1A-resistant S. frugiperda.

In another embodiment, the maize plants show high toxicity to susceptible lepidopteran pests, including but not limited to S. frugiperda.

In one embodiment, the present invention relates to an isolated nucleic acid molecule comprising a nucleotide sequence that is unique to Event ME240913.

In another embodiment, the present invention relates to an isolated nucleic acid molecule that binds an heterologous DNA molecule introduced into the genome of Event ME240913 to the genomic DNA in Event ME240913 comprising at least 10 or more (e.g. 15, 20, 25, 30 or more) contiguous nucleotides of the heterologous DNA molecule and at least 10 or more (e.g. 15, 20, 25, 30 or more) contiguous nucleotides of the genomic DNA flanking the heterologous DNA molecule insertion site. Also included are nucleotide sequences comprising 10 or more nucleotides of the contiguous insertion sequence of Event ME240913 and at least one nucleotide of flanking DNA of Event ME240913 adjacent to the insertion sequence. Such nucleotide sequences are unique and diagnostic to Event ME240913. Nucleic acid hybridization or amplification of the genomic DNA of Event ME240913 produces an amplicon comprising such unique sequences that enable the diagnosis of Event ME240913. In one aspect of this embodiment, the nucleotide sequence is selected from the group consisting of SEQ ID NOs: 4, 5 and 8, and complements thereof.

In another embodiment, the invention relates to an isolated nucleic acid molecule comprising a nucleotide sequence comprising at least one junction sequence from Event ME240913, wherein the junction sequence spans the junction between a heterologous expression cassette inserted into the maize genome and the maize genomic DNA by pairing the insertion site that is unique to said Event ME240913 and is diagnostic of Event ME240913. In one aspect of this embodiment, the junction sequence is selected from the group consisting of SEQ ID NOs: 4 and 5 and complements thereof.

In another embodiment, the present invention relates to an isolated nucleic acid molecule that joins a heterologous DNA molecule to the maize plant genome in Event ME240913, which comprises at least one sequence selected from the group consisting of SEQ ID NOs: 4, 5, and complements thereof.

In another embodiment, the present invention relates to an isolated nucleic acid molecule comprising a nucleotide sequence that is unique to Event ME240913, wherein said nucleotide sequence encodes a protein comprising the amino acid sequence of SEQ ID NO: 3. In one aspect of this embodiment, the nucleotide sequence is SEQ ID NO: 8 and/or the complement thereof.

In another embodiment, the invention relates to an isolated nucleic acid molecule comprising a nucleotide sequence selected from the group consisting of 4, 5 and 8, and complements thereof. In one aspect of this embodiment, the isolated nucleic acid molecule is present in a maize seed deposited with the American Type Culture Collection under accession number PTA-126224, or plants grown from said seed.

In one embodiment of the present invention, an amplicon comprising a nucleotide sequence unique to Event ME240913 is provided. In one aspect of this embodiment, the nucleotide sequence is selected from the group consisting of SEQ ID NOs: 11 and 15 and complements thereof.

In another embodiment, the present invention encompasses flanking sequence primers for detecting Event ME240913. Such flanking sequence primers comprise a nucleotide sequence of at least 10 contiguous nucleotides from the 5′ or 3′ flanking sequence. In one aspect of this embodiment, the contiguous nucleotides are selected from at least 10 contiguous nucleotides of SEQ ID NO: 6 (5′ flanking sequence), or complements thereof. In another aspect of this embodiment, the primer of the 5′ flanking sequence has the sequence of SEQ ID NO: 9 or a complement thereof. In another aspect of this embodiment, contiguous nucleotides are selected from at least 10 contiguous nucleotides of SEQ ID NO: 7 (the 3′ flanking sequence) or complements thereof. In yet another aspect of this embodiment, the primer of the 3′ flanking sequence has the sequence of SEQ ID NOs: 12 or a complement thereof.

In still another embodiment, the present invention encompasses a pair of polynucleotide primers comprising a first polynucleotide primer and a second polynucleotide primer that function together in the presence of a DNA template from Event ME240913 in a sample to produce a diagnostic amplicon for Event ME240913. In one aspect of this embodiment, the first primer and/or the second primer is chosen from SEQ ID NO: 9, 10, or complements thereof. In another aspect of this embodiment, the first primer and/or the second primer is selected from the group consisting of SEQ ID NOs: 13, 14, and complements thereof. In yet another aspect of this embodiment, the amplicon that is produced by the pair of primers comprises SEQ ID NO: 11, 15, or complements thereof.

In another embodiment, the present invention encompasses a pair of polynucleotide primers comprising a first polynucleotide primer and a second polynucleotide primer that function together in the presence of a DNA template from Event ME240913 in a sample to produce a diagnostic amplicon for Event ME240913. Where the first primer is equal to or complementary to a sequence from the maize plant genome that matches the insertion point of a heterologous DNA sequence inserted into the Event ME240913 genome, and the second polynucleotide primer sequence is equal to or is complementary to the heterologous DNA sequence inserted into the genome of Event ME240913.

In one aspect of this embodiment, the first polynucleotide primer comprises at least 10 contiguous nucleotides from position 1-116 and 6307-6424 of SEQ ID NO: 8 and complements thereof. In another aspect of this embodiment, the first primer is selected from the group consisting of SEQ ID NOs: 9, 13, and complements thereof. In another aspect of this embodiment, the second polynucleotide primer comprises at least 10 contiguous nucleotides from position 117-6306 of SEQ ID NO: 8 or complements thereof. In still another aspect of this embodiment, the second polynucleotide primer is selected from the group consisting of SEQ ID NOs: 10, 14, and complements thereof.

In another aspect of this embodiment, the first polynucleotide primer, which is shown in SEQ ID NO: 9, and the second polynucleotide primer, which is shown in SEQ ID NO: 10, work together in the presence of a DNA template of Event ME240913 in a sample to produce a diagnostic amplicon for Event ME240913. In one embodiment of this aspect, the amplicon comprises the nucleotide sequence shown in SEQ ID NO: 11.

In yet another embodiment, the present invention relates to a method for detecting the presence of a nucleic acid molecule that is unique to Event ME240913 in a sample comprising maize nucleic acids, wherein the method comprises: (a) contacting the sample with a pair of primers, (b) performing a nucleic acid amplification reaction to produce an amplicon, and (c) detecting the amplicon.

In another embodiment, the present invention relates to a method for detecting the presence of a nucleic acid molecule that is unique to Event ME240913 in a sample comprising a maize nucleic acid, wherein the method comprises: (a) contacting the sample with a probe that hybridizes under high stringency conditions to genomic DNA from Event ME240913 and does not hybridize under high stringency conditions to DNA from a control maize plant, wherein the probe comprises at least 10 contiguous nucleotides of a nucleotide sequence selected from the group consisting of SEQ ID NO: 4, SEQ ID NO: 5 and complements thereof; (b) subjecting the sample and probe to high stringency hybridization conditions; and (c) detecting hybridization of the probe to the nucleic acid molecule. Detection can be performed by any means well known in the art including fluorescence, chemiluminescence, radiological, immunological and the like. In the case where hybridization is used as a means for amplifying a particular sequence to produce an amplicon that is diagnostic for Event ME240913, production and detection of the amplicon by any means well known in the art is indicative of hybridization with a target sequence where at least one probe or primer is used.

The term “biological sample” defines a sample derived from a maize plant that contains or is suspected to contain a nucleic acid comprising between five and ten nucleotides on either side of the point at which one or the other of the two ends of the inserted heterologous DNA sequence is joined to the genomic DNA sequence within the chromosome into which the heterologous DNA sequence was inserted, in this document also known as junction sequences. Furthermore, the junction sequence comprises as little as two nucleotides: wherein they are the first nucleotide within the flanking or genomic DNA adjacent to the one covalently joined to the first nucleotide within the inserted heterologous DNA sequence. In one aspect of this embodiment, the probe comprises a nucleotide sequence comprising at least 10 contiguous nucleotides of SEQ ID NOs: 4, 5, and complements thereof.

In yet another embodiment, the present invention relates to a kit for detecting nucleic acids that are unique to Event ME240913 in a biological sample. The kit comprises at least one nucleic acid molecule of sufficient length of contiguous polynucleotides to function as a primer or probe in a method for detecting the nucleic acid. Amplification or hybridization to a target nucleic acid sequence in a sample followed by detection of the amplicon or hybridization to the target sequence diagnoses the presence of nucleic acid sequences unique to Event ME240913 in the sample. The kit further comprises other materials required to allow nucleic acid amplification or hybridization. In one aspect of this embodiment, a nucleic acid molecule present in the kit comprises a nucleotide sequence selected from SEQ ID NO: 9, 10, 12, 13, 14, 16, and complements thereof. In another aspect of this embodiment, the nucleic acid molecule is a primer selected from the group consisting of SEQ ID NOs: 9, 10, 13, 14, and complements thereof. In yet another aspect of this embodiment, the amplicon comprises SEQ ID NO: 11, 15, or complements thereof. A variety of detection methods can be used, including but not limited to TAQMAN, thermal amplification, ligase chain reaction, Southern-blot, ELISA, and colorimetric and fluorescent detection methods. In particular, the present invention provides kits for detecting the presence of the target sequence, that is, at least the sequence of SEQ ID NO: 4, 5, or a junction sequence in a sample containing genomic nucleic acid from ME240913. The kit is comprised of at least two polynucleotides capable of binding at or substantially adjacent to the target site and at least one means for detecting binding of the polynucleotide to the target site. The detection means can be fluorescence, chemiluminescence, colorimetry or isotopy and can at least be coupled with immunological methods to detect the binding. The kit can also detect the presence of the target site in a sample, i.e. at least the sequence of SEQ ID NO: 4, 5, or a junction sequence of Event ME240913, taking advantage of two or more polynucleotide sequences that together are capable of binding to nucleotide sequences adjacent to or within approximately 100 base pairs of the target sequence and that can be extended together to form an amplicon that contains at least the target site.

In another embodiment, the present invention relates to a method for detecting Cry1Da protein in a biological sample, the method comprising: (a) extracting tissue protein from Event ME240913; (b) analyzing the extracted protein using an immunological method comprising antibodies specific to the Cry1Da protein produced by Event ME240913; and (c) detecting the binding of said antibody to Cry1Da protein.

In yet another embodiment, the present invention relates to a plant product derived from a maize plant of Event ME240913, tissue, or seed, wherein the plant product comprises a nucleotide sequence that is equal or complementary to the sequence that is unique to Event ME240913, and wherein the sequence is detectable in the plant product using a nucleic acid amplification or hybridization method. In one aspect of this embodiment, the nucleotide sequence is equal or complementary to at least one of SEQ ID NOs: 4, 5 and complements thereof. In another aspect of this embodiment, the plant product is selected from the group consisting of corn flour, corn meal, corn syrup, corn oil, cornstarch and cereals manufactured in whole or in part which containing corn-based products.

In another embodiment, the present invention relates to an extract of a plant product derived from a ME240913 maize plant, tissue or seed comprising a nucleotide sequence that is equal or complementary to a sequence that is unique to ME240913. In one aspect of this embodiment, the sequence is detectable in the extract using a nucleic acid amplification or hybridization method. In another aspect of this embodiment, the sequence is equal to or complementary to at least one of SEQ ID NOs: 4 and 5. Also, in another aspect of this embodiment, the plant product is selected from the group consisting of corn flour, corn meal, corn syrup, corn oil, cornstarch and cereals manufactured in whole or in part containing corn-based products.

Another embodiment of the present invention relates to a maize plant, or parts thereof, and the seeds of a maize plant comprising the genotype of transgenic Event ME240913, wherein the genotype comprises at least one nucleotide sequence of SEQ ID NOs: 4, 5 or complements thereof. One example of corn seed comprises the nucleic acid molecules of the invention that were deposited on Oct. 28, 2019 and assigned accession number PTA-126224. In one aspect of this embodiment, the maize plant is from Hill maize lines. However, one of ordinary skill in the art will recognize that the ME240913 genotype can be introduced into any plant variety that can be bred with maize, including wild-type maize species, and therefore the list of lines reproduced in this way should not be limited.

In another embodiment, the present invention relates to a maize plant comprising at least a first and a second DNA sequences joined to form a contiguous nucleotide sequence, wherein the first DNA sequence is within a junction sequence and comprises at least approximately 10 contiguous nucleotides selected from the group consisting of nucleotides 1-116 and 6307-6424 of SEQ ID NO: 8, and complements thereof, wherein the second DNA sequence is within the inserted heterologous DNA sequence and comprises at least about 10 contiguous nucleotides selected from the group consisting of nucleotides 117-6306 of SEQ ID NO: 8, and complements thereof; and wherein the first and second DNA sequences are useful as probes or nucleotide primers to detect the presence of maize Event ME240913 nucleic acid sequences in a biological sample. In one aspect of this embodiment, nucleotide primers are used in a DNA amplification method to amplify a target DNA sequence from standard DNA extracted from the maize plant and the maize plant is identifiable from other maize plants by producing a amplicon corresponding to a DNA sequence comprising SEQ ID NO: 11, 15 and complements thereof.

In one embodiment, the present invention relates to a maize plant, wherein genotype ME240913 confers on the maize plant resistance against lepidopteran insect pests. In one aspect of this embodiment, the transgenic genotype that confers on the maize plant of the invention resistance to lepidopteran insect pests comprises a truncated or modified cry1 Da gene.

In another embodiment, the maize plant expresses suitable leaf concentrations of truncated cry1Da protein modified to provide high levels of protection to the plant leaf against S. frugiperda damage. In another embodiment, the high level of plant leaf protection was found to occur in several S. frugiperda populations in Brazil, including populations known to have high frequencies of cry1F-resistant S. frugiperda. In another embodiment, insertion of the cry1Da gene from the maize plant ME20913 produces adequate expression of the truncated and modified Cry1Da protein in leaf tissue to produce high toxicity to susceptible lepidopteran species, including S. frugiperda. In yet another embodiment, the maize plant ME20913 is highly toxic to Cry1F resistant S. frugiperda.

In yet another embodiment, the present invention provides a method for producing a maize plant resistant to lepidopteran insect pests comprising the steps of: sexually crossing a first parental maize plant with a second parental maize plant, wherein said first or second parental maize plant comprises DNA of Event ME240913, so as to produce a plurality of first-generation progeny plants; selecting a first-generation progeny plant comprising Event ME240913. Preferably the method further comprises selfing the first-generation progeny plant so as to produce a plurality of second-generation progeny plants; and selecting from the second-generation progeny plants, one plant comprising Event ME240913. In one embodiment, the selection step can be based on the assessment of resistance to lepidopteran insect pests, detection of Event ME240913 DNA according to the methods taught in the present invention or treatment with herbicide and selection of herbicide resistant plants promoted by the herbicide resistance gene PAT (bar) present in Event ME240913. In a preferred embodiment the method for producing a transgenic maize plant comprising the unique nucleic acids of the invention comprises sexually crossing a first parental maize plant containing Event ME240913 with a second non-transgenic parental maize plant to produce progeny plants, selecting a first-generation progeny plant that is resistant to infestation by lepidopteran insect pests, repeating the backcross cycle for 4 times, selfing the parental plant containing Event ME240913 to obtain homozygous plants.

In another embodiment, the present invention provides a method of producing hybrid maize seeds comprising the steps of: planting seeds of a first congenital maize line comprising Event ME240913 and seeds of a second congenital line having a different genotype; sexually crossing the two different congenital lines; and harvesting the hybrid seed thus produced. In a preferred embodiment, the method comprises at least one of the steps of cultivating maize plants resulting from said plantings until the flowering season and emasculating the plant flowers of one of the congenital maize lines. In one aspect of this embodiment, the first bred maize line provides the female descendants. In one aspect of this embodiment, the first bred maize line provides the male descendants. The present invention further relates to hybrid seeds produced by the method of the present invention and hybrid plants grown from the seed.

The following examples have the sole purpose of illustrating one or more preferred embodiments of the invention and are not to be construed as limiting the scope of the invention.

### EXAMPLES

### Example 1—Gene Construct

The gene construct containing the ubiquitin promoter, the nucleotide sequence of SEQ ID NO: 1 that codes for the amino acid sequence of truncated Cry1Da insecticidal protein of SEQ ID NO: 3, optimized for expression in maize and the 3′ region of the Agrobacterium nopaline synthase gene was synthesized at the DNA Cloning Service (http://www.dna-cloning.com/) in the pUC vector flanked by HindIII and EcoRI restriction sites. The construct was transferred from the pUC to the pTF101 vector (Paz et al., 2004) using EcoRI and HindIII restriction enzymes and T4 ligase, according to the manufacturer's instructions (LifeTech). Selection of the recombinant pTF101 UBI::cry1Da::NOS plasmid was performed by transforming E. coli DH5a using spectinomycin and cloning was confirmed by sequencing and cleavage with the HindIII and BamHI enzymes. For sequencing, the commercial kit BigDye Terminator v3.1 (Applied Biosystems) was used. Plasmid DNA from two bacterial colonies containing the gene construct was sequenced and compared with the sequence of interest and they were found to be identical.

Once cloning of UBI::cry1Da::NOS gene into plasmid pTF101 was confirmed, this gene construct was used to transform Agrobacterium tumefaciens EHA101 strain using the electroporation methodology (BioRad/MicroPulser). The same procedure to confirm transformation as above was performed to demonstrate the presence of the binary vector containing the cry1 Da gene in A. tumefaciens. Plasmid DNA was isolated from A. tumefaciens colonies, amplified with primers to detect the bar gene.

Agrobacterium tumefaciens EHA 101 containing the gene constructs of interest (UBI::cry1Da::NOST and 35S::bar::35T) was used for genetically transforming maize.

### Example 2—Genetic Transformation of Immature Hill Maize Embryos Via Agrobacterium tumefaciens

The genotype used in this transformation protocol is Hill maize (Armstrong et al., 1991), according to the protocol by Frame et al. (2002), with minor modifications. Briefly, for transformation of this genotype, immature embryos of between 1.8-2.0 mm in length (10 to 12 days after pollination) were collected. Spikes used to collect the embryos were dipped in a 1:1 solution of commercial bleach (2.5% sodium hypochlorite) and distilled H₂O with 1 to 2 drops of Tween 20, for 20 minutes. Then, they were rinsed with sterile distilled water for 5 minutes, twice.

Immature embryos were collected with the aid of a spatula from a superficial cut of the grains. To transfer the gene construct to maize, Agrobacterium tumefaciens EHA101 was used. From a stock culture of A. tumefaciens containing the gene construct of interest kept in glycerol at −80° C. a streak was made in YEP medium (5 g·L⁻¹ yeast extract; 10 g·L⁻¹ peptone; 5 g·L⁻¹ NaCl; 15 g·L⁻¹ bacto agar) containing the necessary antibiotics (100 mg·L⁻¹ spectinomycin and 50 mg·L⁻¹ kanamycin) and the plate was incubated for 2 to 3 days at 28° C., parent plate). For genetic transformation, Agrobacterium was streaked using a colony isolated from the parent plate in YEP medium containing the necessary antibiotics. The plate was incubated for 2 to 5 days at 19° C. Then, Agrobacterium was resuspended in infection medium (4.0 gL g·L⁻¹ N6 salts; 68.4 g·L⁻¹ sucrose; 36.0 g·L⁻¹ glucose; 0.7 g·L⁻¹ proline; 1.5 mg·L⁻¹ 2,4-D; 1.0 mL·L⁻¹ N6 vitamins (1000×=1.0 g·L⁻¹ thiamine HCl; 0.5 g·L⁻¹ pyridoxine HCl; 0.5 g·L⁻¹ nicotinic acid); pH 5.2) supplemented with 100 μM acetoseringone until OD550=0.3-0.4 was reached and incubated in a shaker at ˜150 rpm, 23° C. for 2 hours.

For infection of immature maize embryos, 50 to 100 embryos were collected in 1 mL of infection medium plus acetoseringone. After collection, the embryos were rinsed twice, 1 mL of the bacterial culture was added, and the suspension incubated for five minutes at 23° C. After infection, the embryos were transferred to the surface of co-culture medium (4.0 g·L⁻¹ N6 salts; 1.5 mg·L⁻¹2.4-D; 30.0 g·L⁻¹ sucrose; 0.7 g·L⁻¹ proline; 1.0 mL·L⁻¹ N6 vitamins (1000×); 0.85 mg·L⁻¹ AgNO₃; 100 μM acetoseringone; 300 mg·L⁻¹ L-cysteine; 3.0 g·L⁻¹ phytagel; pH 5.8) with the scutellum facing upwards. Plates were incubated in the dark at 20° C. for 3 to 5 days. After co-cultivation the embryos were transferred to the resting medium (4.0 g·L⁻¹ N6 salts; 1.5 mg·L⁻¹ 2.4-D; 30.0 g·L⁻¹ sucrose; 0.5 g·L⁻¹ MES; 0.7 g·L⁻¹ proline; 1.0 mL·L⁻¹ vitamins N6 (1000×); 0.85 mg·L⁻¹ AgNO₃; 100 mg·L⁻¹ Tioxin; 3.0 g·L⁻¹ phytagel; pH 5.8) at 28° C. (dark) for 7 to 15 days. Then, the embryos were transferred to the selection medium (4.0 g·L⁻¹ N6 salts; 1.5 mg·L⁻¹ 2.4-D; 30.0 g·L⁻¹ sucrose; 0.5 g·L⁻¹ MES; 0.7 g·L⁻¹ proline; 1.0 mL·L⁻¹ vitamins N6 (1000×); 0.85 mg·L⁻¹ AgNO₃; 100 mg·L⁻¹ Tioxin; 1.5 and 3.0 mg/L Bialaphos; 3.0 g·L⁻¹ phytagel; pH 5.8) (25 embryos/plate). Subcultures of these embryos in selective media are carried out every 15 days to select vigorously growing callus.

Selected calluses were transferred to regeneration medium (4.62 g·L⁻¹ MS salts; 60.0 g·L⁻¹ sucrose; 100 mg·L⁻¹ myo-inositol; 1.0 mL·L⁻¹ MS vitamins (1000×); 1.5 mg/L Bialaphos; 4.0 g·L⁻¹ Phytagel; pH 5.8) and incubated at 26±2° C. (in the dark) for 15 to 21 days. Ready for germination calluses having a dry appearance and opaque white color were transferred to the germination medium (4.62 g·L⁻¹ MS salts; 30.0 g·L⁻¹ sucrose; 100 mg·L⁻¹ myo-inositol; 1.0 mL·L⁻¹ MS vitamins (1000X=0.5 gL⁻¹ thiamine HCl; 0.5 g·L⁻¹ pyridoxine HCl; 0.05 g·L⁻¹ nicotinic acid); 3.0 g·L⁻¹ phytagel; pH 5.8) (12 calluses per plate), 25° C., 80-100 μE/m²/sec of light intensity, 16 hour-photoperiod).

Seedlings with well-developed roots and leaf structures measuring about 5 cm in length (14 to 20 days) were transplanted into pots in a greenhouse containing a commercially available mix of soil and organic matter (⅔ soil and ⅓ organic matter (TDP 30/15) with an intermediate acclimatization stage.

After obtaining the Hill genotype with Event ME240913, the event was introgressed from the Hill genotype to the tropical L3 line using molecular marker-assisted selection.

### Example 3—Bioassays

To assess the susceptibility of transgenic maize expressing the truncated Cry1Da protein to S. frugiperda, bioassays were carried out in the laboratory.

The bioassays were performed as follows: newly hatched S. frugiperda caterpillars were used to infest leaves of transgenic cry1Da maize plants and the non-transgenic isoline (5 caterpillars per plant). Maize development stages used were V7 and V8 and the experiments were carried out in plastic containers and incubated in an acclimatized growth chamber (28C and 60% humidity, light for 12 hrs). Assessments of the damage scores were made after 05 days. In each case, the experimental design consisted of: “experimental group” (Event ME240913 of transgenic maize containing the truncated cry1Da construct) and “control group” (non-transgenic maize).

Evaluated parameters were: injury score using the scale proposed by Carvalho, 1970 (0: plant with undamaged leaves; 1: plant with shaved leaves; 2: plant with perforated leaves; 3: plant with torn leaves; 4: plant showing damage to the cartridge, and 5: plant showing a destroyed cartridge); caterpillar survival (the number of surviving caterpillars in each pot was counted); and caterpillar biomass (using a precision scale of four decimal points).

### Example 4—Bioassays to Control Spodoptera Frugiperda Using Transgenic Maize Event ME240913

S. frugiperda assays: first, Event ME240913 was tested for control of such a pest. Event seeds were germinated in a greenhouse and when the plants reached the stage between the V10 and V12 leaf stage, the two youngest leaves of each plant were used in S. frugiperda bioassays. Three repetitions were performed, five caterpillars per repetition. Hill and L3 maize leaves were used as a negative control (caterpillars grow normally) and Viptera® maize leaves were used as a positive control (caterpillars cannot grow). In this first test, Event ME240913 was found to have good ability to control the caterpillar development reaching 100% mortality (Table 1).

TABLE 1
Evaluation of Event ME240913 in S. frugiperda control

| | Live | | Total weight |
|---|---|---|---|
| | caterpillars | | of live |
| | (After 05 | Dead | caterpillars |
| Event/Repetition | days) | caterpillars | (mg) |
| ME240913 (cry1Da)/1 | 0 | 5 | 0 |
| ME240913 (cry1Da)/2 | 0 | 5 | 0 |
| ME240913 (cry1Da)/3 | 0 | 5 | 0 |
| Viptera Maize | 0 | 5 | 0 |
| Control +/1 | | | |
| Viptera Maize | 0 | 5 | 0 |
| Control +/2 | | | |
| Viptera Maize | 0 | 5 | 0 |
| Control +/3 | | | |
| Hill Maize Control −/1 | 5 | 0 | 70.3 |
| Hill Maize Control −/2 | 4 | 1 | 77.3 |
| Hill Maize Control −/3 | 5 | 0 | 68.7 |
| L3 Maize Control −/1 | 4 | 1 | 39.5 |
| L3 Maize Control −/2 | 5 | 0 | 84.4 |
| L3 Maize Control −/3 | 5 | 0 | 61.6 |

The bioassay with this event was repeated, using four replicates with 20 caterpillars per replicate, and the results confirmed that this event has the ability to control the development of S. frugiperda (Table 2). FIG. 3 is representative of the S. frugiperda feeding bioassays in non-transgenic maize and in the transgenic maize of the present invention. In this experiment, treatment with the Viptera maize genotype was not used.

TABLE 2
Evaluation of transgenic maize events compared to the S.
frugiperda control (bioassay 2)

| | | | Total weight of |
|---|---|---|---|
| | Live | Dead | live caterpillars |
| Event | caterpillars | caterpillars | (mg) |
| ME240913 (cry1Da)/1 | 0 | 20 | 0 |
| ME240913 (cry1Da)/2 | 0 | 20 | 0 |
| ME240913 (cry1Da)/3 | 0 | 20 | 0 |
| ME240913 (cry1Da)/4 | 0 | 20 | 0 |
| Hill/1 | 18 | 02 | 176.7 |
| Hill/2 | 18 | 02 | 131.7 |
| Hill/3 | 15 | 05 | 137.0 |
| Hill/4 | 17 | 03 | 212.7 |
| L3/1 | 19 | 01 | 131.9 |
| L3/2 | 19 | 01 | 132.5 |
| L3/3 | 19 | 01 | 133.3 |
| L3/4 | 18 | 02 | 115.6 |

### Example 5—Exposure to Fresh Leaf Tissue from Two Genetic Backgrounds Containing Event ME240913

In this experiment, Helix L85 line X Embrapa L3 ME240913 line hybrid 1 was used (leaves V8 and V9). Hybrid 2 was Hill ME240913 X Embrapa L3 line (V5 and V6 leaves) and the control was a Helix L85 line X Embrapa L3 line hybrid (V8 and V9 leaves). Both hybrids were heterozygous to Event ME240913.

Discs of fresh leaves of 1.8 cm in diameter were cut using a metal cutter and placed in 2.0% agar (1 mL/well) in 128-well plastic bioassay trays (Bio-Ba-128, CD International, Pitman, N.J., USA).

A susceptible, standard laboratory population of fall armyworm, S. frugiperda from Embrapa Milho e Sorgo was used in the tests, the same population used by Omoto et al 2016 as a susceptibility standard(Omoto, C., Bernardi, O., Salmeron, E., Sorgatto, R. J., Dourado, P. M., Crivellari, A., Carvalho, R. A., Willse, A., Martinelli, S., & Head, G. P. (2016). Field-evolved resistance to Cry1Ab maize by Spodoptera frugiperda in Brazil. Pest Management Science, 72 (9), 1727-1736.). maintained on an artificial diet and without any insecticidal or Bt selective pressure.

A newly hatched larva (0 to 24 hours) was placed in each well containing a leaf disc using a fine brush. Plates were sealed with Bio-CV-16 adhesives (C-D International, Pitman, N.J., USA) and placed in an acclimatized chamber (temperature of 26±1° C.; relative humidity of 60±10%; 14:10h light:dark photoperiod). One hundred and twenty larvae were used for each treatment.

Mortality was assessed after 24 hours of exposure and then daily until 100% mortality. Dead caterpillars were considered to be those that did not respond to the touch of the brush.

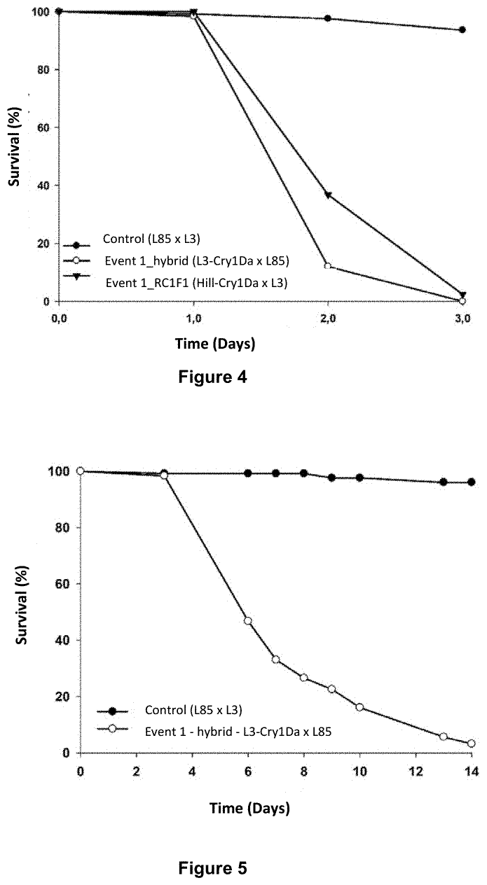

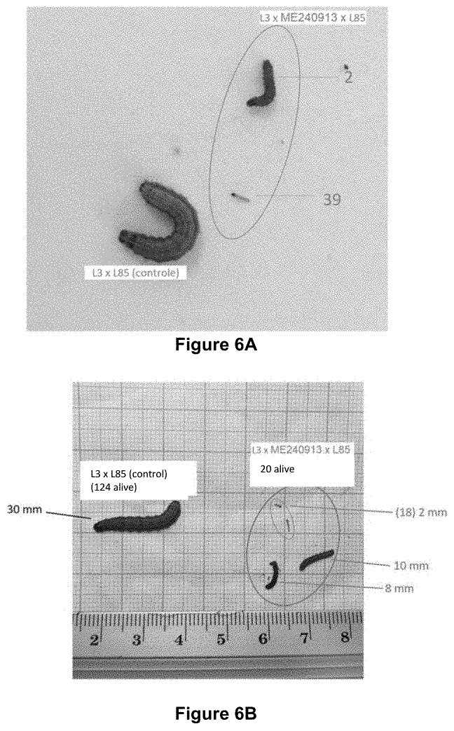

Fresh maize leaves expressing leaf tissue from Event ME240913 caused S. frugiperda mortality from day two after feeding and 100% mortality after three days. The same pattern of rapid and complete mortality was observed in the two hybrids tested. The result is shown in FIG. 4.

### Example 6—Exposure to Leaf Lyophilized Tissue of Event ME240913 Diluted at 1:25 in the Artificial Diet

This experiment assessed Event ME240913 in the breeding between the Helix L85 X Embrapa L3 lines containing Event ME240913 (leaves harvested at the V8 and V9 stages) and as a control the hybrid of Helix L85 X Embrapa L3 lines (leaves V8 and V9). Results were compared with those obtained using leaves of the control hybrid Helix L85 X Embrapa L3, also harvested at V8 and V9 stages.

About seven plants of the two maize hybrids described above were harvested after 27 days of growth. Leaves between the V8 and V9 development stages were placed in plastic bags frozen with liquid nitrogen and transferred to an ultra-low freezer at −80° C. Leaf tissues were lyophilized using freeze-drying. After lyophilization, the material was ground using a tissue grinder (IKA A11 Basic). Samples of lyophilized and ground leaves were stored in plastic cups with sealed lids at room temperature.

Lyophilized transgenic maize leaves were prepared at a 1:25 ratio in the artificial diet for S. frugiperda at 4% (w/w). Negative control contained 4% lyophilized non-transgenic tissue. About 1 L of S. frugiperda artificial diet was prepared with an adapted protocol containing only 56% of the total amount of agar to the regular protocol. The diet was cooled and kept at 55° C. in a water bath as required. For each treatment, 160 g of S. frugiperda diet was added to the plastic cup containing pre-weighed lyophilized tissue. The sheet tissue was mixed with a spatula until visually uniform. The mixture was transferred to a thick plastic bag with a hole at one end and the diet was added to each of the wells in a bioassay tray (128-well CD-International trays) by pressing the diet through the entire bag (as a pastry bag). About 0.8 ml of diet/leaf powder mixture was dispensed into each of 128 individual wells for each transgenic and non-transgenic material (total of 256).

The standard susceptible S. frugiperda population was used, as described in the aforementioned test.

A newly hatched larva (0 to 24 hours) was placed in each of the wells using a fine brush. The plates were sealed with Bio-CV-16 adhesives (CD International, Pitman, N.J., USA) and placed in an acclimatized chamber (temperature 26±1° C.; relative humidity 60±10%; 14:10 h light:dark photoperiod).

Mortality was recorded on days 3, 6-10 and 13-14 days after exposure. Visibly inactive larvae that did not move when touched by a fine brush were considered dead.

In this experiment, a 67% mortality was observed on day 7 and 97% on day 14 (125/128 larvae). The three remaining larvae showed significant growth inhibition compared to larvae fed on control (control) leaf tissue.

FIG. 5 illustrates the result of survival of newly hatched S. frugiperda caterpillars (%), as assessed up to 14 days after exposure to lyophilized leaves at a 1:25 ratio in artificial diet obtained from leaves of Event ME240913 and the control

### Example 7—Protection Against Leaf Damage in Maize Plants of Event ME240913 in Fields Infested by Six Different S. frugiperda Populations from Different Origins

Control plants were obtained from the hybrid between Helix L85 X Embrapa L3 lines. The event-containing plants were obtained from the hybrid between Helix L85 x Embrapa L3 ME240913 lines.

Naturally occurring S. frugiperda populations were collected at the larval stage in six different maize production sites in Brazil, as described: two populations were collected in the State of Parana (Palotina and Ivatuba), two in the State of Mato Grosso (Rondonopolis and Campo Verde) and two in the State of Minas Gerais (Paracatu and Sete Lagoas). Larvae were kept under laboratory conditions with an artificial diet until adulthood (cycle around ˜30 days) without any selective pressure from insecticides or Bt. The newly hatched larvae were then infested on plants at the V4 growth stage under field conditions.

Treatments consisted of a combination of two hybrids and six insect populations with three replications. Five-meter-long plots of five rows each were planted; infestation of these plots was carried out in the three central rows, which were used for the assessment. The two remaining rows were used as a buffer zone (border).

Assessment of the damage caused by the caterpillar feeding on the maize plants was made according to Davis et al. 1992. In summary, a scale from 0 to 9 was used, where 0 represented plants with no damage and 9 plants with destroyed expanded leaves. Thus, unit increase in the scale represented higher levels of leaf damage.

Field scores were recorded at 7, 14 and 21 days after exposure.

The average visual damage of leaves from the control treatment using the maize hybrid L85XL3 produced an infestation score for the six different S. frugiperda populations ranging from 3.4 to 5.19 in the control treatment and close to zero in the Cry1Da-expressing hybrid. That is, the average scores of plants assessed in the hybrid containing the event with Cry1Da were considerably smaller (<0.1).

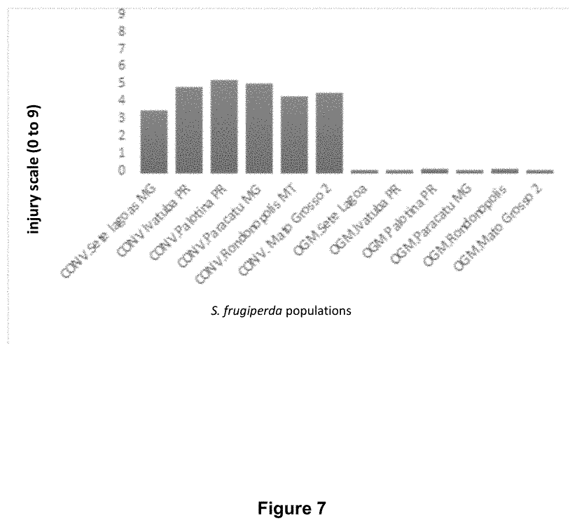

FIG. 7 illustrates damage score results (Davis et al, 1992 scale) from 0 to 9, caused by feeding different S. frugiperda populations (±Confidence Interval, at 5% probability) on a conventional hybrid (cony) that does not express Event ME240913 and on maize hybrids ME240913) in the field. Assessment was made at 14 days after infestation.

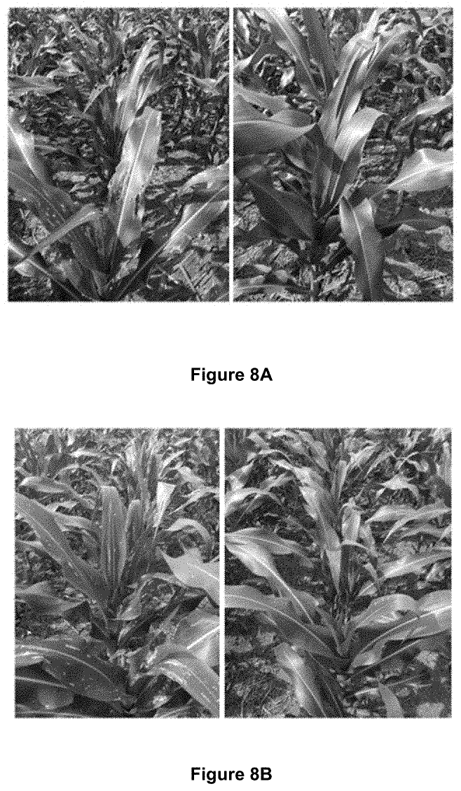

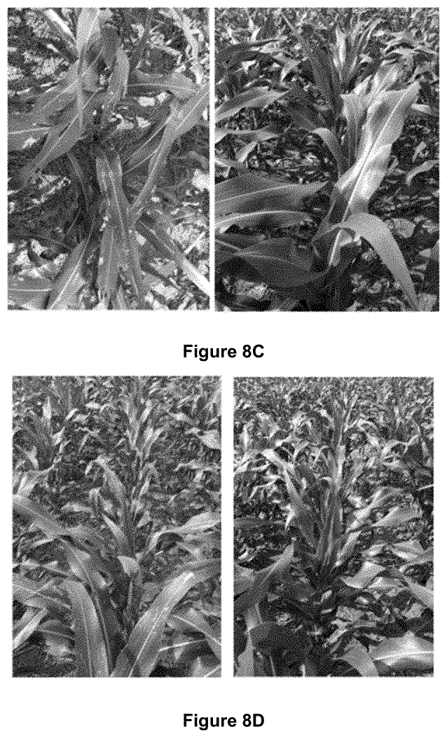

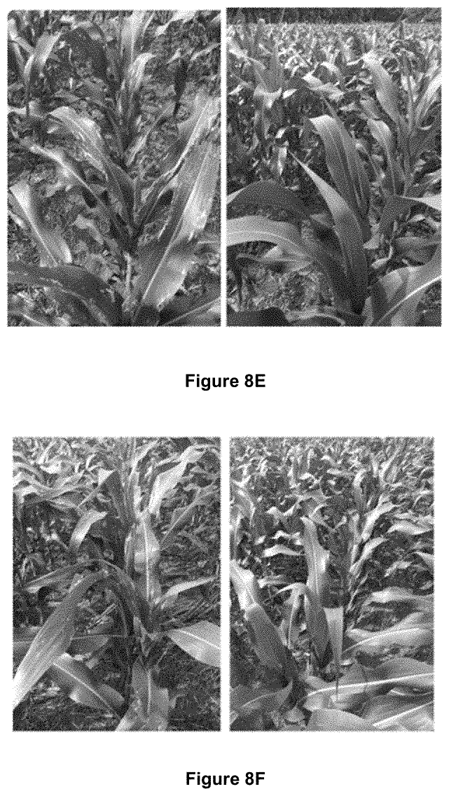

FIGS. 8A to 8F show the single control (conventional) hybrid obtained from the bred between the Helix-L85 x Embrapa-L3 lines (on the left in the photos) and its respective isogenic version containing the event, Helix-L85 x Embrapa-L3 with Event ME240913 (on the right in the photos). Each photo shows the conventional version and the version containing Event ME240913 individually infested by each of the six different S. frugiperda populations collected in Brazil, at the following sites: population collected in Palotina-PR (8A), Rondonópolis-MT (8B)), Rondonopólis+Campo Verde-MT (8C), Paracatu-MG (8D), Sete Lagoas-MG (8E) and Ivatuba-PR (8F). Leaf damage is not visible in the controls on the left in each photo, while the hybrid that contains Event ME240913 shows no damage (hybrid on the right in each photo).

### Example 8—Bioassays Using Caterpillar Populations Resistant to Cry1F Gene-Containing Transgenic Maize

Assays were performed to verify the potential of Event ME240913 in controlling a S. frugiperda population resistant to Cry1F protein.

The experiment was carried out in a greenhouse by planting transgenic maize containing the Event ME240913 and non-transgenic corn (negative control). Newly hatched caterpillars belonging to two distinct S. frugiperda populations (a population resistant to Cry1F gene and another population sensitive to the same gene) were inoculated on maize plants (15 caterpillars per plant) at V7 and V8 stages. After infestation, the pots were isolated with a voil cage and damage assessment was made after 07, 14 and 21 days. The experimental design consisted of 04 treatments, with 05 pots each, containing from 02 to 03 maize plants per pot:

Treatment 1: Transgenic event ME240913 infested with a S. frugiperda population resistant to Cry1F protein according to Leite et al., 2016.

Treatment 2: Non-transgenic isogenic L3 line infested with the Cry1F-resistant S. frugiperda population according to Leite et al 2016.

Treatment 3: Transgenic Event ME240913 infested with the population of susceptible caterpillars reared and maintained in the entomology laboratory of Embrapa Milho e Sorgo.

Treatment 4: Non-transgenic isogenic line infested with the population of susceptible caterpillars reared and maintained in the entomology laboratory of Embrapa Milho e Sorgo.

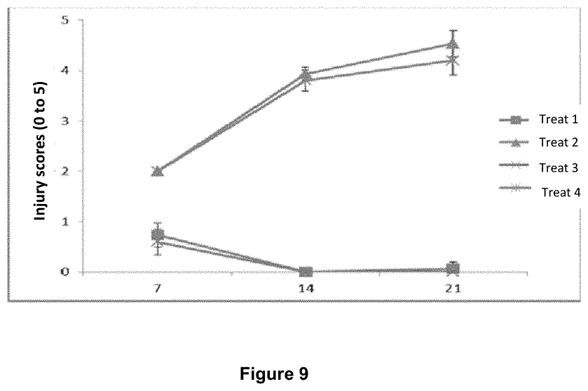

Results have shown that the transgenic plant comprising the Event ME240913 was able to control infestation with Cry1F protein resistant-S. frugiperda population so effectively as the susceptible population by inhibiting its development (FIG. 9) and protecting the plant from the attack by such a pest, as observed by the injury score (±CI, P=0.05). Survival percentage of S. frugiperda, as assessed 21 days after release of the caterpillars under different treatments, was 0% for treatments 1 and 3, and about 65% and 35% for treatments 2 and 4, respectively. S. frugiperda biomass, as assessed 21 days after the caterpillars were placed under different treatments, was 0% for treatments 1 and 3, and about 260 mg and 300 mg for treatments 2 and 4, respectively. In both cases, the non-overlapping CI averages differ from each other (P=0.05).

Example 8 describes the use of a codon-optimized Cry1Da sequence to produce maize plants expressing a truncated Cry1Da sequence exhibiting high toxicity level (100% mortality) for both wild-type and Cry1F-resistant S. frugiperda populations. The fact of identifying 100% mortality in S. frugiperda populations resistant to Cry1F when fresh leaves from Event ME240913 were used to feed these S. frugiperda populations confirms the fact that the truncated and codon modified protein expressed from the Cry1Da gene acts through a different mechanism than that existing in the commercial event that contains the Cry1F gene.

### Deposit of Biological Material

Maize seeds from Event ME240913 disclosed above were deposited on Oct. 28, 2019 in accordance with the Budapest Treaty with the American Type Culture Collection (ATCC), 1801 University Boulevard, Manassas, Va. 20110, under accession number PTA-126224.

1. A nucleic acid molecule, characterized by comprising a nucleotide sequence that is unique to Event ME240913, wherein the nucleotide sequence is selected from the group consisting of SEQ ID NO: 4, SEQ ID NO: 5, and complements thereof.

2. The nucleic acid molecule according to claim 1, characterized in that the nucleotide sequence encodes a truncated Cry1Da protein comprising the amino acid sequence of SEQ ID NO: 3.

3. The nucleic acid molecule according to claim 1, characterized in that the nucleic acid molecule is comprised in a maize seed deposited with the American Type Culture Collection under accession number PTA-126224.

4. An amplicon, characterized in that it comprises the nucleic acid molecule, as defined in claim 1.

5. A pair of polynucleotide primers, characterized by comprising a first polynucleotide primer and a second polynucleotide primer which function together in the presence of an Event ME240913 DNA template in a sample to produce a diagnostic amplicon for Event ME240913, wherein the first polynucleotide primer recognizes a nucleotide sequence selected from the group consisting of SEQ ID NO: 6, SEQ ID NO: 7, and complements thereof, and wherein the second polynucleotide primer recognizes the nucleotide sequence of SEQ ID NO: 2, or the complement thereof.

6. The transgenic maize plant, cell, or tissue thereof, characterized by comprising a nucleic acid molecule as defined in claim 1.

7. The maize plant, according to claim 6, characterized in that the maize plant is resistant to lepidopteran insect pests.

8. The maize plant according to claim 6, characterized in that it provides high level of protection to maize plant leaves against Lepidoptera pests, including, among others, S. frugiperda, including S. frugiperda populations resistant to Cry1F and/or Cry1A.

9. The maize plant according to claim 6, characterized in that the leaf tissue thereof has high toxicity to susceptible Lepidopteran pests, preferably Spodoptera frugiperda.

10. A maize seed, characterized in that it comprises the nucleic acid molecule, as defined in claim 1.

11. A maize seed, characterized in that it is deposited with the American Type Culture Collection under accession number PTA-126224.

12. A transgenic maize plant, characterized in that it is produced from a seed as defined in claim 10.

13. A plant product, characterized in that it is derived from the maize plant as defined in claim 6.

14. The plant product according to claim 13, characterized in that it is selected from the group consisting of corn flour, cornmeal, corn syrup, corn oil, cornstarch, and cereals made in whole or in part of corn products.

15. A method for detecting the presence of a nucleic acid molecule that is unique to Event ME240913 in a sample comprising maize nucleic acids, characterized in that it comprises:
(a) contacting the sample with a pair of primers as defined in claim 5;
(b) performing a nucleic acid amplification reaction to produce an amplicon; and
(c) detecting the amplicon.

16. A method for detecting the presence of a nucleic acid molecule that is unique to Event ME240913 in a sample comprising maize nucleic acids, characterized by comprising:
(a) contacting the sample with a probe that hybridizes under high stringency conditions to genomic DNA from Event ME240913 and does not hybridize under high stringency conditions to DNA from a control maize plant, wherein the probe comprises a nucleotide sequence selected from the group consisting of SEQ ID NO: 4, SEQ ID NO: 5, and complements thereof;
(b) subjecting the sample and probe to high stringency hybridization conditions; and
(c) detecting hybridization of the probe to the nucleic acid molecule.

17. A kit for detecting nucleic acids that are unique to Event ME240913, characterized by comprising at least one nucleic acid molecule which is a primer or probe comprising a nucleic acid sequence which, by amplifying or hybridizing a target nucleic acid sequence in a sample followed by detection of the amplicon or hybridization to the target sequence are diagnostic for the presence of nucleic acid sequences unique to Event ME240913 in the sample.

18. A method for the production of a maize plant comprising Event ME240913 that is resistant to lepidopteran insect pests, characterized by comprising:
sexually crossing a first parental maize plant with a second parental maize plant, wherein said first or second parental maize plant comprises the DNA of Event ME240913, so as to produce a plurality of first-generation progeny plants;
selecting from the progeny plants, a plant comprising the DNA of Event ME240913.

19. The method for the production of hybrid maize seeds, characterized by comprising:
planting seeds of a first congenital maize line comprising a nucleotide sequence selected from the group consisting of SEQ ID NO: 4, SEQ ID NO: 5, and complements thereof and seeds of a second congenital line having a different genotype;
sexually crossing the two different congenital lines with each other; and
harvesting the hybrid seed thus produced.

20. The method according to claim 19, characterized in that the plant of the first congenital maize line is the female parent.

21. The method according to claim 19, characterized in that the plant of the first congenital maize line is the male parent.

22. A hybrid seed, characterized in that it is produced by the method as defined in claim 19.

23. A maize plant, characterized in that it is produced by growing the hybrid maize seed as defined in claim 20.

24. A method for controlling lepidopteran insect pests on maize plants, wherein the maize plants comprise a nucleic acid molecule comprising a nucleotide sequence selected from the group consisting of SEQ ID NO: 4, SEQ ID NO: 5, and complements thereof, characterized by comprising planting seeds obtained from a plant comprising said nucleic acid molecule in a growing area of maize plants susceptible to lepidopteran insect pests.

25. The method according to claim 24, characterized in that the lepidopteran insect pest is S. frugiperda.

26. The method according to claim 25, characterized in that S. frugiperda is resistant to Cry1F and/or Cry1A.

27. The method according to claim 25, characterized in that fresh plant leaves containing DNA from Event ME240913 are highly toxic to S. frugiperda.

28. Use of a plant, plant cell, plant part or seed comprising a nucleic acid molecule, as defined in claim 1, characterized in that it is for breeding with a second plant, regenerating a plant, planting or growing a field of plants or producing a plant product.
